(19) United States
(12) Patent Application Publication
(10) Pub. No.: US 2005/0165458 A1
(43) Pub. Date: Jul. 28, 2005

(54) Method and system to provide therapy for depression using electroconvulsive therapy(ECT) and pulsed electrical stimulation to vagus nerve(s)

(76) Inventors: Birinder R. Boveja, Milwaukee, WI (US); Angely Widhany, Milwaukee, WI (US)

Correspondence Address: BIRINDER R. BOVEJA & ANGELY WIDHANY, P. O. BOX 210095, MILWAUKEE, WI, 53221, US

(21) Appl. No.: 11/086,526

(22) Filed: Mar. 22, 2005

Related U.S. Application Data
(63) Continuation-in-part of application No. 10/196,533, filed on Jul. 16, 2002.
Application No. 10/196,533 is a continuation of application No. 10/142,298, filed on May 9, 2002.

(51) Int. Cl. A61N 1/18

(52) U.S. Cl. 607/45

(57) ABSTRACT

A method and system for providing therapy or alleviating the symptoms of depression (including bipolar depression, unipolar depression, severe depression, treatment resistant depression, and melancholia), by providing electroconvulsive therapy (ECT) to the brain and pulsed electrical stimulation to the vagus nerve(s) for afferent neuromodulation. ECT is provided via two electrodes placed on the head, either in the unilateral or bilateral configuration. Constant-current (or constant-voltage) stimuli are provided using brief-pulsed outputs at frequencies between 30 Hz and 100 Hz. The transcranial stimuli delivered are strong enough to induce seizures. Pulsed electrical stimulation to the vagus nerve(s) may be provided continuously in ON-OFF repeating cycles. The two electrical stimulation therapies (ECT and VNS) may be given in any order, any combination, or any sequence as determined by the physician. The two electrical stimulation therapies may also be used with or without pharmaceutical therapy. Pulsed electrical vagus nerve stimulation (VNS) may be provided using an implanted pulse generator (IPG) or an external stimulator used in conjunction with an implanted stimulus-receiver. In one aspect of the invention the pulse generator system may comprise communication capabilities for networking over a wide area network, for remote interrogation and programming.

This application is a continuation of application Ser. No. 10/196,533 filed Jul. 16, 2002, entitled “METHOD AND SYSTEM FOR MODULATING THE VAGUS NERVE (₁₀th CRANIAL NERVE) USING MODULATED ELECTRICAL PUSES AND AN INDUCTIVELY COUPLED STIMULATION SYSTEM”, which is a continuation of application Ser. No.10/1 42,298 filed on May 09, 2002. The prior applications being incorporated herein in entirety by reference, and priority is claimed from these applications.

### FIELD OF INVENTION

This invention relates to providing electrical therapy to the body, more specifically using a combination of electroconvulsive therapy (ECT) to the brain and providing electrical pulses to vagus nerve(s), to provide therapy for severe depression.

### BACKGROUND

This patent application is directed to providing electroconvulsive therapy (ECT) and vagus nerve stimulation/blocking with electrical pulses to provide therapy for, or to alleviate symptoms of severe depression. Both electroconvulsive therapy (ECT) and pulsed electrical stimulation of vagus nerve(s) have shown clinical utility for severe depression, when other treatments such as psychotherapy and antidepressant medications have failed. Shown in conjunction with FIG. 1 is a depiction of the methodology of the invention, where a combination of ECT and pulsed electrical stimulation to vagus nerve(s) are applied to provide therapy for severe depression.

ECT is given under anesthesia and with muscle relaxants. The electrical charge, which lasts 1 to 4 seconds, produces a short seizure that lasts 30 to 60 seconds. The seizure induced by ECT helps treat depression. ECT treatments are usually repeated 2 to 3 times a week for 2 to 3 weeks. Pulsed electrical stimulation to the vagus nerve(s) 54 is supplied using a pulse generator means and a lead with electrodes in contact with nerve tissue. Vagus nerve(s) stimulation is typically applied 24 hours/day, 7 days a week, in repeating cycles. This patent application is directed to combined use of ECT and VNS, which may be used in addition to any drug therapy. The dose of electrical therapy (ECT and VNS), and sequence of delivery is at the discretion of the physician. This would be particularly useful for depression (including bipolar depression, unipolar depression, severe depression, treatment resistant depression, melancholia) and other neuropsychiatric disorders.

### BACKGROUND OF DEPRESSION

Depression is a very common disorder that is often chronic or recurrent in nature. It is associated with significant adverse consequences for the patient, patient's family, and society. Among the consequences of depression are functional impairment, impaired family and social relationships, increased mortality from suicide and comorbid medical disorders, and patient and societal financial burdens. Depression is the fourth leading cause of worldwide disability and is expected to become the second leading cause by 2020.

Among the other currently available treatment modalities include, pharmacotherapy with antidepressant drugs (ADDs), specific forms of psychotherapy, and phototherapy. ADDs are the usual first line treatment for depression. Commonly the initial drug selected is a selective serotonin reuptake inhibitor (SSRI) such as fluoxetine (Prozac), or another of the newer ADDs such as venlafaxine (Effexor).

Several forms of psychotherapy are used to treat depression. Among these, there is good evidence for the efficacy of cognitive behavior therapy and interpersonal therapy, but these treatments are used less often than are ADDs. Phototherapy is an additional treatment option that may be appropriate monotherapy for mild cases of depression that exhibit a marked seasonal pattern.

Many patients do not respond to initial antidepressant treatment. Furthermore, many treatments used for patients who do not respond at all, or only respond partially to the first or second attempt at antidepressant therapy are poorly tolerated and/or are associated with significant toxicity. For example, tricyclic antidepressant drugs often cause anticholinergic effects and weight gain leading to premature discontinuation of therapy, and they can by lethal in overdose (a significant problem in depressed patients). Lithium is the augmentation strategy with the best published evidence of efficacy (although there are few published studies documenting long-term effectiveness), but lithium has a narrow therapeutic index that makes it difficult to administer; among the risks associated with lithium are renal and thyroid toxicity. Monoamine oxidase inhibitors are prone to produce an interaction with certain common foods that results in hypertensive crises. Even selective serotonin reuptake inhibitors can rarely produce fatal reaction in the form of a serotonin syndrome.

Physicians usually reserve electroconvulsive therapy (ECT) for treatment-resistant cases or when they determine a rapid response to treatment is desirable. When used alone, ECT is also associated with significant risks: long-lasting cognitive impairment following ECT significantly limits the acceptability of ECT as a long-term treatment for depression. Furthermore, there is a high percentage of relapse rate, if pharmacological therapy is not administered. Therefore, there is a compelling unmet need for non-pharmacological well-tolerated and effective long-term or maintenance treatments for patients who do not respond well to ECT, or for patients who can not sustain a response to first-line pharmacological therapies.

FIG. 2 (shown in table form) generally highlights some of the advantages and disadvantages of various forms of nonpharmalogical interventions for the treatment of depression. For example, deep brain stimulation is regionally very specific which is positive, but on the other hand requires very invasive surgical procedure. As another example, ECT has clinical applicability in the short run, but on the other hand is associated with long-lasting cognitive impairments. Considering the advantages and disadvantages of different existing treatments, as shown in conjunction with FIG. 2, a combination of ECT therapy and pulsed electrical vagus nerve stimulation is an ideal combination for device based interventions, with or without concomitant drug therapy. Furthermore, in this unique combination, ECT induces stimulation from outside, and vagus nerve stimulation (VNS) approaches the stimulation of centers in brain from inside, as shown in conjunction with FIG. 1. FIG. 3 shows a simplified overall structure of the brain, and FIG. 4 depicts anatomically the relationship between vagus nerve, nucleus of solitary tract, and rest of the brain.

Vagus nerve stimulation, has beneficial effects to the brain, via projections of Solitary Track Nucleus to the different centers in the brain. This is depicted in a simplified block diagram shown in FIG. 5.

Based on this thinking as shown in conjunction with Table 2, which highlights that ECT and vagus nerve stimulation as an ideal combination of nonpharmalogical interventions, with or without concomitant drug therapy.

TABLE 2
Nonpharmacological interventions for the treatment of Depression

| | Regionally | Clinically | |
|---|---|---|---|
| Intervention | specific | applicable | Invasive |
| Electroconvulsive | ++ (+++ if | ++++ | ++ (anesthesia, |
| therapy (ECT) | induced by | | generalized seizure) |
| | magnets) | | |
| Vagus nerve | ++ | +++ | +++ (surgery for |
| stimulation | | | generator implant) |

The initiation and delivery of these two interventions may be in any sequence or combination, and may be in addition to any drug therapy. For example, a patient implanted with vagal nerve stimulator may be given ECT therapy, or alternatively a patient receiving ECT therapy may be implanted with a vagus nerve stimulator. Of course, this may be in addition to any drug therapy that may be given to a patient. It is an object of this invention to provide an optimal device based therapy for depression by supplementing ECT with VNS. ECT provided alone usually has cognitive adverse effects. Advantageously, not only would the cognitive adverse effects be reduced, but the efficacy would also be significantly improved by the combination of ECT and VNS as disclosed in this application.

### PRIOR ART

Prior art is generally directed either to electroconvulsive therapy (ECT) or to vagus nerve stimulation.

U.S. Pat. No. 5,269,302 (Swartz et al) is generally directed to monitoring patient seizures. In the method of his patent, the ECT device includes a special purpose electromyograph (EMG) to detect isolated muscle activity, an electrocardiograph(ECG) to detect heart-beat intervals, and an electroencepyhalograph (EEG) system to detect an EEG parameter of the electrically induced EEG seizure. There is no disclosure or even suggestion for combining ECT with vagus nerve stimulation to provide therapy for depression.

U.S. Pat. No. 4,480,969 (Swartz) is mearly directed to electrode application system and method for electroconvulsive therapy

U.S. Pat. No. 5,871,517 (Abrams) is generally directed to monitoring the extent of therapeutic value of the treatment.

U.S. Pat. No. 5,299,569 (Wernicke et al.) is directed to the use of implantable pulse generator technology for treating and controlling neuropsychiatric disorders including schizophrenia, depression, and borderline personality disorder.

U.S. Pat. No. 6,205,359 B1 (Boveja) and U.S. Pat. No. 6,356,788 B2 (Boveja) are directed to adjunct therapy for neurological and neuropsychiatric disorders using an implanted lead-receiver and an external stimulator.

### SUMMARY OF THE INVENTION

A novel method for providing therapy or alleviating the symptoms of depression (including bipolar depression, unipolar depression, severe depression, treatment resistant depression, melancholia) by providing electroconvulsive therapy (ECT) to the brain and afferent neuromodulation of the vagus nerve(s) with electrical pulses. The combination of ECT and vagus nerve stimulation (VNS) provides a more ideal combination for device based interventions, with or without concomitant drug therapy. In this novel method of therapy, ECT induces stimulation from the outside, and selective vagus nerve stimulation approaches the stimulation from inside the brain.

Accordingly in one aspect of the invention, method and system to provide therapy for, or alleviate the symptoms of severe depression, comprises providing ECT to the brain of a patient and afferent neuromodulation of vagus nerve(s) with electrical pulses.

In another aspect of the invention, the combination of ECT provided to the brain and electrical pulses provided to vagus nerve(s) are in any sequence or any combination, as determined by the physician.

In another aspect of the invention, vagus nerve pulsed electrical stimulation is provided to patients that have received ECT in the past.

In another aspect of the invention, vagus nerve pulsed electrical stimulation is provided to patients who are currently receiving ECT, and drug therapy.

In another aspect of the invention, ECT therapy is provided using brief-pulsed outputs, at frequencies between 30 Hz to 100 Hz.

In another aspect of the invention, the ECT stimuli may be constant-current or constant voltage.

In another aspect of the invention, the afferent modulation of the vagus nerve(s) is by providing electric pulses at any point along the length said vagus nerve(s).

In another aspect of the invention, the system to provide electrical pulses to the vagus nerve(s) has both implanted and external components, and may be one selected from the following group: a) an implanted stimulus-receiver with an external stimulator; b) an implanted stimulus-receiver comprising a high value capacitor for storing charge, used in conjunction with an external stimulator; c) a programmer-less implantable pulse generator (IPG) which is operable with a magnet; d) a programmable implantable pulse generator (IPG); e) a microstimulator; f) a combination implantable device comprising both a stimulus-receiver and a programmable IPG; and g) an IPG comprising a rechargeable battery.

In yet another aspect of the invention, the system for providing electrical pulses to the vagus nerve(s) can be remotely interrogated or remotely programmed over a wide-area network, either wirelessly or over land-lines.

Various other features, objects and advantages of the invention will be made apparent from the following description taken together with the drawings.

### BRIEF DESCRIPTION OF THE DRAWINGS

For the purpose of illustrating the invention, there are shown in accompanying drawing forms which are presently preferred, it being understood that the invention is not intended to be limited to the precise arrangement and instrumentalities shown.

FIG. 1 is a diagram depicting the concept of the invention, where a patient receives electroconvulsive therapy (ECT), and pulsed electrical stimulation to vagus nerve(s) with an implanted stimulator.

FIG. 2 depicts in table form, the peculiarities of different forms of device based therapies for neuropsychiatric disorders

FIG. 3 is a diagram showing the overall structure of the brain.

FIG. 4 is a schematic diagram of the brain showing relationship of vagus nerve and solitary tract nucleus to other centers of the brain.

FIG. 5 is a simplified block diagram illustrating the connections of solitary tract nucleus to other centers of the brain.

FIG. 6A is a diagram showing the placement of electrodes on the head for unilateral ECT.

FIG. 6B is a diagram showing the placement of electrodes on the head for bilateral ECT.

FIG. 7 shows an example of well developed EEG seizure

FIG. 8 depicts heart rate and blood pressure changes with ECT.

FIG. 9 shows the four EEG seizure phases.

FIG. 10 shows the EEG delta activity 24 hours after ECT.

FIG. 11A shows the pulse train to be transmitted to the vagus nerve.

FIG. 11B shows the ramp-up and ramp-down characteristic of the pulse train.

FIG. 12 is a simplified block diagram depicting supplying amplitude and pulse width modulated electromagnetic pulses to an implanted coil.

FIG. 13 depicts a customized garment for placing an external coil to be in close proximity to an implanted coil.

FIG. 14 shows coupling of the external stimulator and the implanted stimulus-receiver.

FIG. 15 is a schematic diagram of the implantable lead.

FIG. 16 is a schematic diagram showing the implantable lead and one form of stimulus-receiver.

FIG. 17 is a schematic block diagram showing a system for neuromodulation of the vagus nerve, with an implanted component which is both RF coupled and contains a capacitor power source.

FIG. 18 is a simplified block diagram showing control of the implantable neurostimulator with a magnet.

FIG. 19 is a schematic diagram showing implementation of a multi-state converter.

FIGS. 20A-C depicts various forms of implantable microstimulators

FIG. 21 is a figure depicting an implanted microstimulator for providing pulses to vagus nerve.

FIG. 22 is a diagram depicting the components and assembly of a microstimulator.

FIG. 23 shows functional block diagram of the circuitry for a microstimulator.

FIG. 24 is a simplified block diagram of the implantable pulse generator.

FIG. 25 is a functional block diagram of a microprocessor-based implantable pulse generator.

FIG. 26 shows details of implanted pulse generator.

FIG. 27 is a diagram showing the two modules of the implanted pulse generator (IPG).

FIG. 28A depicts coil around the titanium case with two feedthroughs for a bipolar configuration.

FIG. 28B depicts coil around the titanium case with one feedthrough for a unipolar configuration.

FIG. 28C depicts two feedthroughs for the external coil which are common with the feedthroughs for the lead terminal.

FIG. 28D depicts one feedthrough for the external coil which is common to the feedthrough for the lead terminal.

FIG. 29 shows a block diagram of an implantable stimulator which can be used as a stimulus-receiver or an implanted pulse generator with rechargeable battery.

FIG. 30 is a block diagram highlighting battery charging circuit of the implantable stimulator of FIG. 29.

FIG. 31 is a schematic diagram highlighting stimulus-receiver portion of implanted stimulator of one embodiment.

FIG. 32A depicts bipolar version of stimulus-receiver module.

FIG. 32B depicts unipolar version of stimulus-receiver module.

FIG. 33 depicts power source select circuit.

FIG. 34A shows energy density of different types of batteries.

FIG. 34B shows discharge curves for different types of batteries.

FIG. 35 depicts externalizing recharge and telemetry coil from the titanium case.

FIGS. 36A and 36B depict recharge coil on the titanium case with a magnetic shield in-between.

FIG. 37 shows in block diagram form an implantable rechargable pulse generator.

FIG. 38 depicts in block diagram form the implanted and external components of an implanted rechargable system.

FIG. 39 depicts the alignment function of rechargable implantable pulse generator.

FIG. 40 is a block diagram of the external recharger.

FIG. 41 depicts an implantable system with tripolar lead for selective unidirectional blocking of vagus nerve(s) stimulation

FIG. 42 depicts selective efferent blocking in the large diameter A and B fibers.

FIG. 43 is a schematic diagram of the implantable lead with three electrodes.

FIG. 44 depicts remote monitoring of stimulation devices.

FIG. 45 is an overall schematic diagram of the external stimulator, showing wireless communication.

FIG. 46 is a schematic diagram showing application of Wireless Application Protocol (WAP).

FIG. 47 is a simplified block diagram of the networking interface board.

FIGS. 48A and 48B is a simplified diagram showing communication of modified PDA/phone with an external stimulator via a cellular tower/base station.

### DETAILED DESCRIPTION OF THE INVENTION

The following description is of the best mode presently contemplated for carrying out the invention. This description is not to be taken in a limiting sense, but is made merely for the purpose of describing the general principles of the invention. The scope of the invention should be determined with reference to the claims.

In the method and system of this invention, adjunct therapy is provided for severe and treatment resistant depression, by providing a combination of electro-convulsive therapy (ECT) and pulsed electrical stimulation to vagus nerve(s). This device based intervention may be in addition to any drug therapy. The delivery of ECT and vagus nerve(s) stimulation (VNS) may be in any order, any combination, or any time sequence. The dose of electrical therapy whether for ECT, or for VNS, is of course dependent on the attending physician, and may be titrated. Advantageously, VNS pulses may also be remotely controlled over a wide area network as disclosed later in this application.

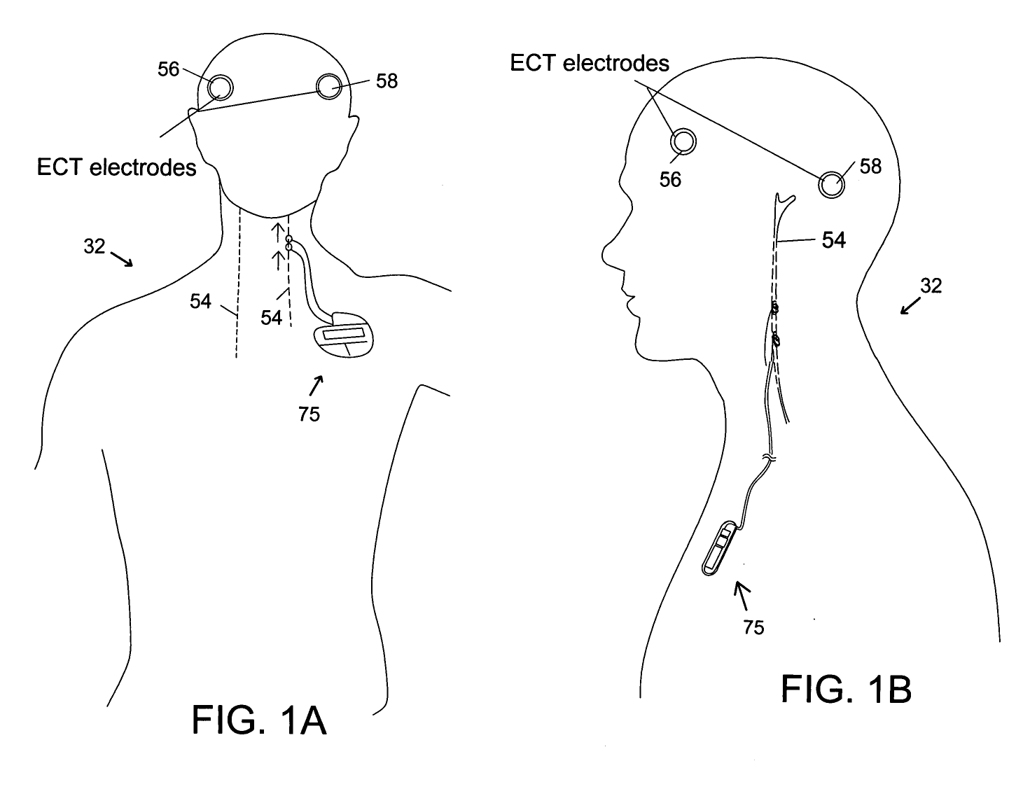

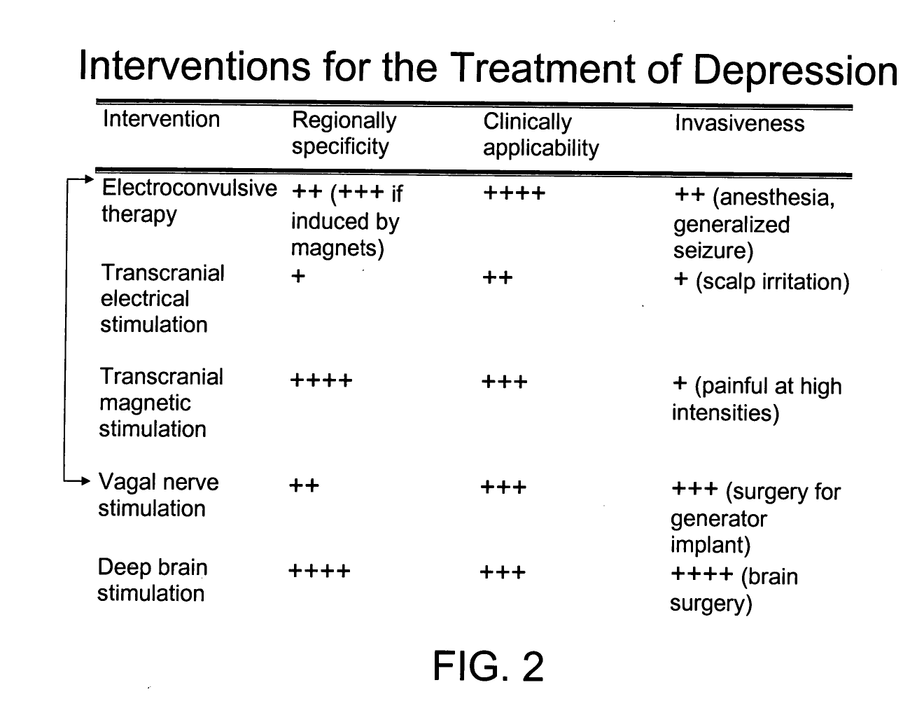

For patients with more severe forms of major depression (variously designated psychotic, endogenous, suicidal, delusional, or melancholic), ECT is one of the very effective treatments available. A substantial body of experimental evidence supports the use of ECT in the treatment of depression. Against this backdrop, not much significant improvement in the therapeutic potency of antidepressant drugs has materialized since the introduction of imipramine and amitriptyline nearly a half-century ago. ECT is used because of its demonstrable efficacy, safety, and relative ease of administration, all due in large measure, to the advances in technique (e.g., succinylcholine muscle relaxation, barbiturate anesthesia, oxygenation, unilateral and bifrontal electrode application, seizure monitoring, brief- and ultrabrief-pulse stimulation) that have been introduced over the years. But, ECT is not a cure for difficult depressive episodes, and is associated with cognitive impairments. Therefore, to supplement ECT, whereby increasing efficacy and decreasing the side effects of device therapy, as depicted in conjunction with FIG. 1, a combination use of ECT and pulsed electrical stimulation to vagus nerve(s), utilizing implanted and external components is disclosed here.

It is known that repeated production of generalized CNS seizures is required to produce the clinical benefits of ECT. Thus the goal of an ECT treatment session is to induce a generalized seizure of “adequate” duration in the CNS. Subconvulsive electrical stimuli or those inducing only partial (focal) seizures have no therapeutic benefit. Similarly, treatments in which seizures are terminated immediately following stimulation are ineffective.

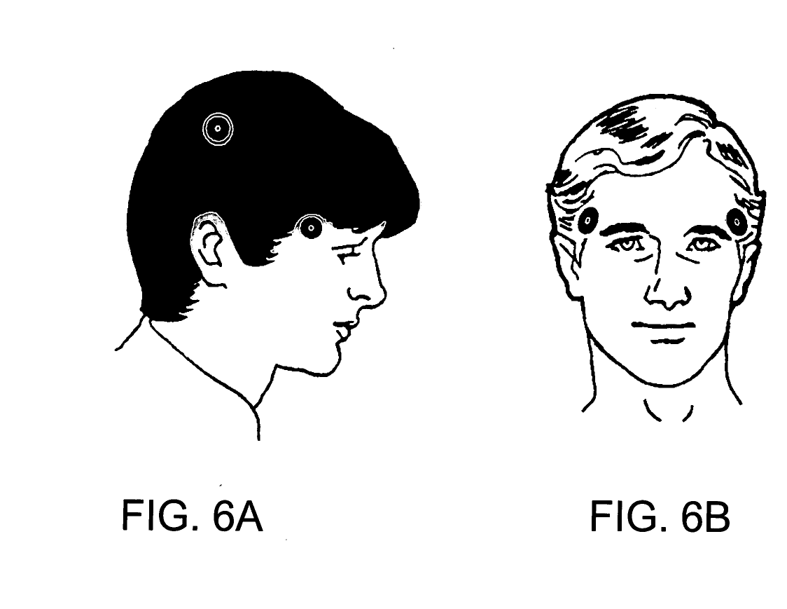

As shown in conjunction with FIG. 6, to provide therapy, typically one of two electrode placements are used for ECT; a unilateral, nondominant hemisphere placement or bilateral placement. The electrodes are usually either hand-held devices or metal plates contained in a band that is affixed to the patient's head. Hand-held electrodes are easier to position, but the position may change as pressure is applied to form a good electrical contact. Several varieties of unilateral placement can be used, but most practitioners prefer a temporoparietal position. For all right-handed patients, the electrodes are placed over the right cerebral hemisphere. Most (more than 60 percent) of left-handed patients are either left hemisphere dominant for language or have mixed dominance, so a right hemisphere placement is appropriate for these patients as well. For bilateral treatments, electrodes are usually positioned bitemporally; other placements are experimental.

Prior to positioning the electrodes, the skin must be carefully prepared to improve electrical contact and diminish interelectrode impedance. This is done by cleaning the electrode area with a saline-soaked pad and coating the electrodes with a conducting gel. Measurement of the patient's skin (static) impedance before administering the electrical stimulus for ECT provides important information on the quality of the skin-to-electrode contact: If the skin is oily, or if the electrodes are applied loosely or with inadequate conductive gel, a high impedance will be registered, informing the physician that his technique requires improvement.

Such impedance testing is performed with a high frequency, very low milliamperage current that is undetectable by the patient. The static impedance is much higher than the dynamic impedance that is recorded during the actual passage of the treatment stimulus; The dynamic impedance is function of the summed electrical properties of the skin, hair, scalp, subcutaneous tissues, periosteium, bone, dura and pia mater, brain, blood vessels, blood and cerebrospinal fluid, and falls dramatically during the passage of the treatment stimulus.

Impedance has both static and dynamic components. In the ECT circuit, most of the static impedance to current flow is across the skull (approximately 18,000 ohms/cm). While the impedance to current flow across the skin and through brain tissue is only about 200 ohm/cm. When an electrical stimulus is applied via electrodes placed on the patient's head, the low-impedance pathway is along the skin between the electrodes. Thus, most of the stimulus is shunted between the stimulating electrodes and little (<20 percent) enters the cranial cavity to stimulate the nervous system. The closer the treatment electrodes are placed to each other (e.g., as for bifrontal or unilateral ECT), the greater this shunt will be. The charge entering the brain is then distributed along the paths of least impedance. With bitemporal ECT, current densities are greatest in the frontal poles, diminishing in more remote areas in proportion to the square root of the distance traversed; with unilateral ECT, current density is greatest in the pathway between the electrodes, across the surface of the brain. Impedance to the electrical stimulus during ECT is primarily attributable to the patient, although corrosion may cause substantial impedances to develop in the stimulus leads delivering the current, and their connectors.

Excitable tissues are stimulated by the flow of current (or more properly by the movement of ions across the cell membrane). Current (I) is the amount of charge (Q) measured in coulombs flowing per unit time (t). Thus, I=Q/t. The force that drives current flow is the applied electrical field (measured in volts [V]). The relationship between current and voltage is of course given by Ohm's law: V=I×R, where R is the resistance to current flow measured in ohms. An ECT treatment involves the application of electricity as an alternating current (AC); the resistance term of the circuit is more properly described as impedance (Z). Impedance includes the DC resistance as well as terms for capacitance (the ability to store charge on conductors that are separated by an insulator) and inductance (the ability to induce a voltage across the tissue). The ability to store charge on either side of the lipid bilayer is a fundamental biophysical property of cell membranes; thus capacitance is important to the ECT circuit.

ECT stimulation devices typically have only one of two outputs: they are either current generators or voltage generators. Constant-current stimulation is more physiological and more preferred method of the two for inducing neuronal depolarization. Constant-current is also more likely to induce a seizure in the presence of a high impedance because of insufficient current delivery with constant voltage or constant energy devices. Most ECT devices presently used are constant-current stimulators. A constant-current device also ensures stable delivery of the stimulus over a wide range of impedances, in contrast to constant voltage or energy, which more readily induce brief or missed seizures when administered close to the patient's threshold. A second type of ECT stimulator uses a constant voltage source. For the purposes of this invention, ECT equipment from any manufacturer may be used, including Somatics Inc. (Lake Bluff, Ill.), Maeta Corp. (Tualtin, Oreg.), and Medcraft Corp. (Darien, Conn.).

In contrast to a constant-voltage source, in a constant-current stimulator, the applied current is independent of the impedance between the electrodes. According to Ohm's law, the applied voltage varies directly with the impedance. Thus, when the impedance between the electrodes is high, the applied voltage from a constant-current stimulator can become very high (sometimes exceeding 500 Volts, depending on the maximal output of the stimulator). Because the power (measured in watts) dissipated between the electrodes is a product of current and voltage (P=IV, or P=I²R), significant risk of local tissue damage exists if the impedance between the electrodes is too great. With most constant-current stimulators, the operator is required to perform a self-test prior to stimulating the patient. This test administers a low-amplitude current to test the interelectrode impedance.

If the impedance exceeds a limit deemed safe, the test fails. In the case of a failed self-test, improved contact between the electrodes and the skin usually lowers impedance. Somewhat counterintuitively, when impedance between the ECT electrodes is too low, induction of convulsions with constant-current stimulator can be more difficult. The difficulty with low-impedance seizure induction develops because the applied voltage becomes too low to drive significant current flow through the high resistance of the skull.

In these devices, the current varies with the resistance between the electrodes; high impedance can cause difficulty inducing seizures because the current flow is too low. Constant-current stimulators offer the advantage of easier quantification of the electrical stimulus. Because current is fixed, the amount of charge (coulombs) administered is simply a product of the current and the time during which current flows (Charge [Q]=I×t), with a constant-voltage stimulator, calculations of administered charge require information about impedance.

The charge passing through the brain is related to the impedance of the head in a complex fashion. Most of the impedance is across the skull, estimated at 18,000 ohms/cm, compared with about 200 ohm/cm across the skin or brain. Although the charge with a constant current device does not vary with impedance, its distribution among the 3 compartments of scalp, skull, and brain does vary with the voltage. At low voltages there is insufficient electromotive force to drive enough current through the high-impedance skull to induce a seizure; most of it is shunted (short-circuited) between the electrodes via the low-impedance scalp. As voltage increases, more and more current penetrates the skull to enter the brain, increasing the likelihood of depolarizing enough neurons to exceed the threshold for a seizure.

There is an inverse relation for constant-current devices between seizure threshold (the charge required to induce a seizure of specified duration) and dynamic impedance. It results in the counterintuitive observation that the high-threshold patients in whom seizures are the most difficult to elicit are actually those with the lowest impedances. This is due to greater shunting of the stimulating current through extracranial tissues, resulting in a lower dynamic impedance and less current entering the brain.

The waveform of the output is another important characteristic of ECT stimulators. Older ECT devices were sine-wave generators. At a frequency of 60 cycles per second (60 Hz), each half sine wave lasts 8.3 ms with a significant stimulus flowing about 75 percent of this time. A basic property of neuronal action potentials, the cellular activity driving generalized seizures, is a duration of a few milliseconds. Furthermore, following an action potential there is a period of several milliseconds during which it is either impossible or relatively difficult to fire a second action potential (the absolute and relative refractory periods). During a sine-wave stimulus, much of the current flow occurs during inexcitable periods. Thus, sine waves tend to drive neuronal firing rather inefficiently. A constant-step stimulus applied for a long period of time is even more inefficient. A device that administers repeated brief pulses (0.5 to 2.0 milliseconds) of current to trigger action potential firing at rates similar to the intrinsic firing patterns of neurons in critical regions of the CNS is preferred. The benefits of brief-pulse stimuli compared with step pulses or sine waves have been documented in experimental preparations. Evidence suggests that pulses less than 0.5 millisecond in duration (referred to as “ultrabrief” pulses) are likely to be ineffective ECT stimuli.

ECT stimulators using brief-pulse outputs, typically at frequencies of 30 to 100 Hz are the preferred mode, because when brief-pulse outputs are given with a constant-current generator, it is relatively easy to quantify the electrical stimulus. The administered charge is calculated by adding the total time that brief pulses are applied and multiplying this duration by the pulse amplitude. In most constant-current stimulators each cycle consists of one positive and one negative pulse. Thus, the calculation of stimulus duration (D) is given by: D=pulse width×pulse frequency 2× train duration. Most constant-current stimulators used in the United States have maximal charge outputs of 500 to 600 mC. Assuming an interelectrode impedance of 200 ohms, this output translates into a stimulus energy of less than 100 joules (waft-seconds). Because stimulus energy requires information about the interelectrde impedance and impedance measures must take into account both static and dynamic factors, it has been preferable to quantitate ECT stimuli in units of charge rather than units of energy.

Brief-pulse devices deliver a constant-current, so the voltage varies directly with the dynamic impedance of the patient. Because extremely high impedances would draw correspondingly high voltages to maintain the same current across the electrodes, thus markedly increasing the energy generated, brief-pulse devices also limit the maximum voltage that can be applied to about 500 volts.

The features of the electrical stimulus interact with the mode of stimulus to play a role in the therapeutic benefits of ECT. When treatments are administered with electrodes placed bitemporally (FIG. 6), minimally suprathreshold electrical doses produce significant clinical benefits. However, treatment with nondominant hemisphere unilateral electrode placement at stimuli minimally above the seizure threshold produces only marginal clinical improvement, despite inducing what appear to be generalized seizures of adequate duration. The benefits of unilateral ECT increase significantly when electrical doses at least 2.5 times the seizure threshold are used. Based on scientific studies it appears that that the degree to which the electrical stimulus exceed the seizure threshold is critical in determining therapeutic effects of unilateral treatments. The electrical dose plays a role in the cognitive adverse effects of ECT. Higher stimulus intensities are associated with greater memory impairment. Thus, attention to seizure threshold is a major concern for use of ECT.

Seizure threshold is defined empirically as the minimum amount of electrical charge that induces a generalized CNS seizure. There is some debate concerning the proper length of a threshold seizure and whether duration should be measured by electroencephalgram (EEG) or by motor seizure in an isolated limb. Some use a cutoff of 25 seconds, but this limit is arbitrary. Across grouped patient samples, there is a great variability in the mean threshold values obtained for unilateral ECT, for example-ranging from 13 mC to 113mC—which reflects differences in peak current, age, sex, treatment electrode placement, seizure duration criteria and measurement method, electrical stimulus parameters, and the strength of the initial and incremental dosages of the titration schedule. Seizure thresholds tend to be higher in men than in women and higher in older patients than in younger patients. Age-related differences may reflect differences in skull density as well as plasticity of an aging nervous system. Electrode placement also plays a major role, with bilateral (bitemporal) placements having a higher threshold than non-dominant hemisphere placements. Other variable include the patient's electrolyte and hydration status as well as concomitant use of CNS-active medication. ECT has anticonvulsant effects, so recent treatment with ECT can influence threshold measurements. The most important determinant of seizure threshold with current stimulators is pulse duration and frequency.

If an electrical stimulus depolarizes a sufficient number of neurons, a generalized, paroxysmal, cerebral seizure ensues, the threshold for which is defined as the electrical dose (in millicoulombs, mC) that produced it. Subconvulsive stimuli elicit only an electroencephalographic (EEG) “arousal” response of low-voltage fast activity that is indistinguishable in appearance from that seen in the earliest phases of ECT-induced seizures, and has been dubbed the “epileptic recruiting” stage. With substantially suprathreshold stimuli, this initial low-voltage, 18-22-Hz activity is rapidly replaced by a crescendo of high-voltage 1 0-to 20-Hz hyper-synchronous polyspikes occurring simultaneously throughout the brain and corresponding to the tonic phase of the motor seizure. This discharge gradually decreases in frequency as the seizure progresses, evolving into the characteristic polyspike and slow-wave complexes of the clonic motor phase, which slow to 1 to 3 Hz just before seizure termination, and are often abruptly replaced by EEG flattening (“postictal suppression”).

Several electrical dosing schedules may be used for estimating seizure threshold. Typically these dosing regiments begin with a low electrical charge (e.g., 25 mC); increases in the charge are delivered according to a predetermined plan until a generalized seizure is induced. In clinical setting, threshold titration involving a minimal number of stimulation (four or five) are preferred to diminish the risks associated with titration. The last stimulation in the titration series is given at maximal charge. About 30 seconds are allowed between stimulation to ensure that the prior stimulus has not produced a seizure. When the stimulus is near threshold, onset of a generalized seizure may be delayed for several seconds.

It is the induced cerebral seizure, more than any other aspect of the treatment, that is responsible for the fully developed therapeutic effect of ECT. Seizure monitoring is also done to protect from the risks of undetected prolonged seizures. Although direct electrical stimulation of the brain may itself have antidepressant properties, clinical research shows that there is little doubt that the cerebral seizure is central to the therapeutic process, especially in the more severe forms of depression.

Because the EEG directly measures the brain's electrical activity, it remains the primary technique for measuring seizures. Two analog methods are typically incorporated in ECT instruments for amplifying and presenting unprocessed EEG activity during ECT. One uses a chart-drive and penwriter to record the EEG signal on paper; the resulting record is then read by the clinician (or a computer program) as it is generated to determine the occurrence, duration, and end-point of the induced seizure. A second method provides an auditory representation of the EEG signal in the form of a tone that fluctuates with the frequency of the seizure activity and becomes a constant when the seizure ends. This method is as reliable as the first and correlates highly with it; it has been used successfully to detect prolonged seizures requiring termination with benzodiazepines.

Although electrical stimulation of the brain in the absence of a seizure has well-documented therapeutic effects in some forms of depression. It is the much larger effect of the induced seizure that is generally acknowledged to be the primary therapeutic agent of ECT, especially in the more severe forms of depression (like melancholic). It is desirable that a fully developed, bilateral, grand mal seizure is obtained during each treatment session, with ictal characteristics. The seizures should last for 20-30 seconds. An average ECT seizures lasts from 30 to 90 seconds. But, even seizures shorter than 15 seconds can have a therapeutic impact if given with a high enough stimulus dose. Typically, what is sought is a synchronous EEG seizure pattern with high amplitude relative to baseline, well-developed, polyspike and spike-and-slow-wave phases, a clear ictal end-point with pronounced postictal suppression, and a substantial tachycardia response.

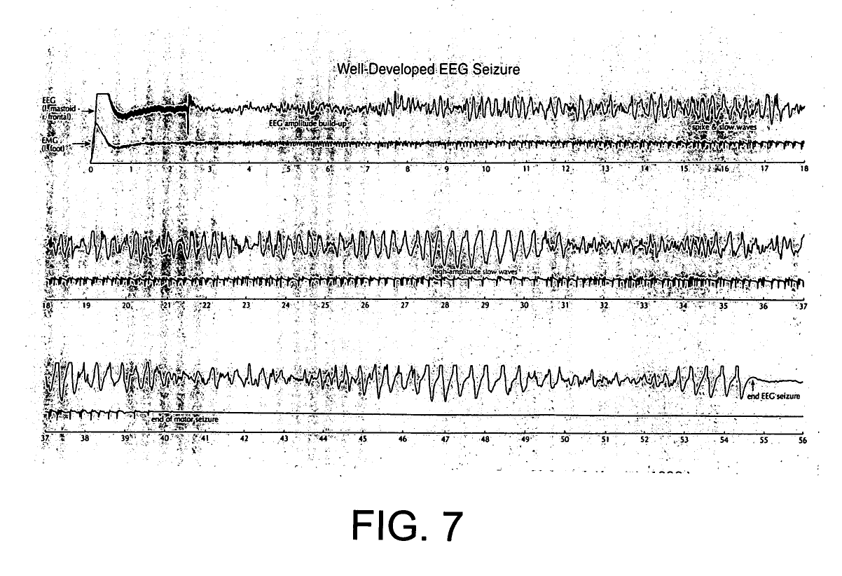

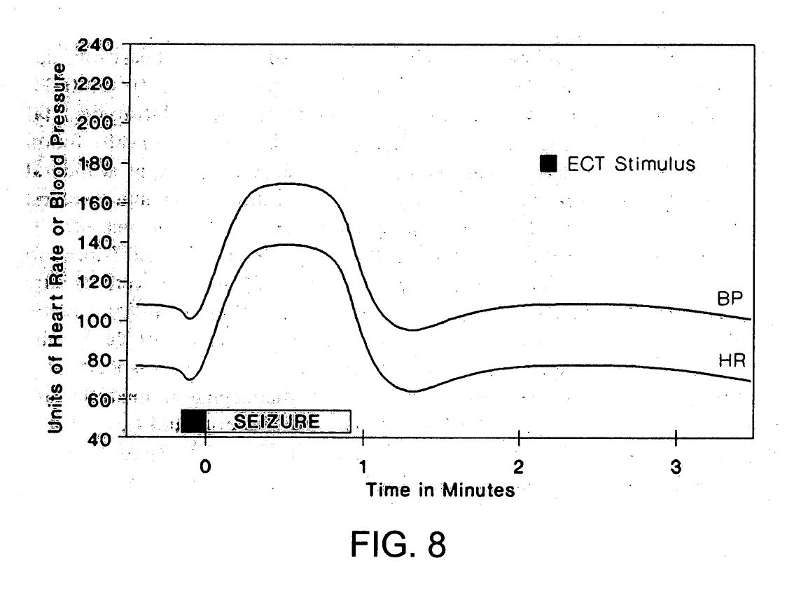

There is consensus of clinical expert opinion that clinically effective stimulation for ECT results in morphologically well-developed, symmetrical, synchronous, high-amplitude seizure activity that is followed by marked post-ictal suppression, an example of which is shown in FIG. 7, and which is accompanied by a prominent tachycardia response (shown in FIG. 8)—phenomena that all reflect increased intracerebral seizure intensity or generalization (e.g., more rapid development and spread) and therefore more effective, seizures.

A sympathoadrenal tachycardia then supervenes, an example is shown in FIG. 8, which is initially driven predominantly by direct sympathetic neural outflow of discharging cardioaccelerator areas in the hypothalamus, descending ipsilaterally by way of the medulla, upper thoracic cord, paravertebral stellate ganglia, and postganglionic cardiac nerves to the heart as described by Berne and Levy in 1981. Adrenal medullary catecholamine release later in the seizure is supposed to contribute to maintaining heart rate above baseline during the late ictal and postictal phases, although the mean duration of the maximal phase of the ECT-induced tachycardis is significantly shorter than that of total paroxysmal EEG seizure activity.

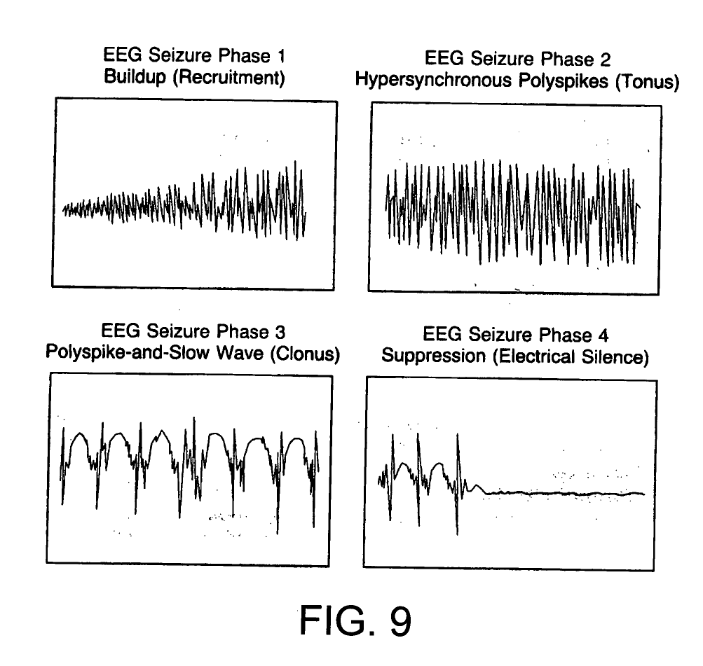

It is the induced cerebral seizure, more than any other aspect of the treatment, that is responsible for the fully developed therapeutic effect of ECT, and from the risks of undetected, prolonged seizures. As shown in conjunction with FIG. 9, electroencephalographic monitoring consistently reveals a progression through a series of characteristic patterns: Build-up, hypersynchronous polyspikes during tonus, and polyspike-and-slow-wave complexes during clonus that terminate in suppression. The approaching end of the seizure is indicated by progressive slowing of the spike-and-wave bursts of clonus. A classical seizure end point occurs when these are abruptly replaced by electrical silence. A distinct end point is also signaled by sudden replacement of paroxysmal clonic activity with lower amplitude, mixed frequencies.

Following a single ECT, very little EEG change persists after the seizure patterns have terminated and been gradually replaced by the pretreatment rhythms. As the numbers of treatments increase, however, the EEG slowing persists into the postconvulsive period, accumulating as a function of the total number of ECTs and their rate of administration. This EEG activity increases in amplitude and duration and decreases in frequency with each additional treatment as long as the rate of administration remains above 1 per week. These changes are accompanied by a decreased mean frequency and total beta activity and an increased mean EEG amplitude, total power, and total proxysmal activity.

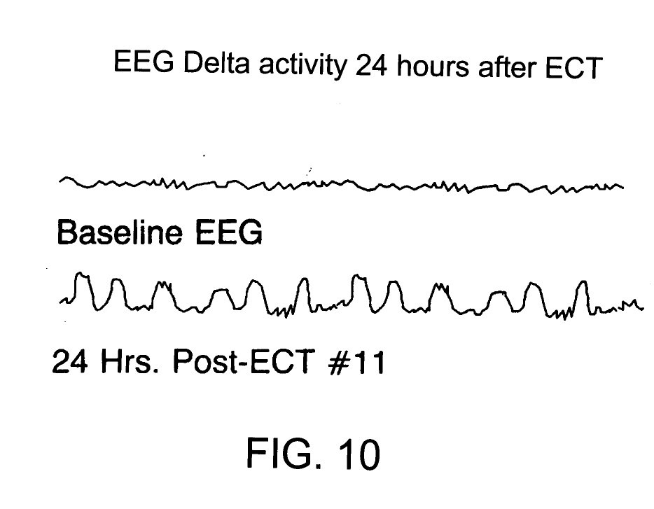

With the usual three treatments per week, the EEG obtained 24 to 48 hours after 6 to 8 seizures given with sine-wave bitemporal ECT is often dominated by theta/delta activity (shown in FIG. 10) with a marked reduction in the abundance of normal alpha/beta rhythms. This postconvulsive (interictal) EEG slowing is also related to the pretreatment EEG, age, and method of seizure induction. Following the final treatment of a course of ECT, the cumulative EEG slowing typicaly diminishes gradually over time and eventually disappears. Most studies show a return to baseline by 30 days post-ECT.

ECT has anticonvulsant properties, and over a course of treatments, seizure threshold increases and seizure duration decreases. Seizures lasting less than 25 seconds are considered less therapeutic than longer seizures yet are associated with the risks and adverse effects of longer seizures. When seizures routinely last less than 25 seconds, several approaches can be used to lengthen them. First, vigorous hyperventilation prior to and during the seizure can lengthen seizures in some patients by diminishing carbon dioxide levels. Second, any medications that raise seizure threshold and that can be withheld safely should be discontinued; these include benzodiazpines, antidepressants, and anticonvulsants given for psychiatric indication. Third, consideration should be given to the dose and type of anesthetic drug. High doses of barbiturates clearly have anticonvulsant effects. Thus, either lowering the dose of barbiturate or changing the anesthetic to etomidate or ketamine can lengthen seizures in some patients. Alternatively, the dose of barbiturate can be significantly lowered (to 20 to 30 mg) and alfentanil (0.25 ug/kg) added to the regimen. Fourth, intravenous administration of caffeine (250 to 1000 mg) significantly prolongs seizure activity in most patients. Theophylline has effects similar to those of caffeine but has been associated with status epilepticus during ECT.

Sustained improvement in psychiatric symptoms rarely occurs with a single ECT treatment. Most, if not all, patients require a course of repeated treatments. A typical course of ECT consists of 6-12 treatments administered two or three times per week over a period of several weeks until improvement in target clinical symptoms reaches a plateau. The total number of treatments administered to a patient in a single treatment course is a function of the diagnosis, rapidity of response, response to any previous course of ECT, severity of illness, and the quality of the response to treatments already received. There have been several attempts to speed this course of treatment by inducing multiple seizures in succession at a single treatment session (often referred to as “multiple-monitored ECT”).

Because few illnesses are permanently relieved by a brief exposure to a therapeutic agent, most medical treatments consist of an acute phase followed by a maintenance phase. Maintenance drug therapy with lithium or tricyclic antidepressants after a successful course of ECT substantially reduces these relapse rates. A typical schedule for maintenance ECT provides a treatment 1 week after the initial course is successfully completed, a second in 2 weeks, a third in 3 weeks, and the fourth and subsequent treatments at monthly intervals for up to 6 months. Some patients may not remain well on monthly interval maintenance ECT and will require treatments at 3-week intervals or, rarely, biweekly. This latter spacing should only be given with unilateral ECT, for 2 to 3 consecutive treatments, before again attempting to decrease the seizure frequency.

Cognitive adverse effects of ECT show great individual variability. For example, some patients have little recollection of ECT procedure while other can describe in detail all events up to the time they lose consciousness. The reasons for this variability are not certain. It is also hypothesized, based on clinical research that pulsed electrical stimulation to vagus nerve(s) would alleviate some of the cognitive adverse effects.

Most patients experience a period of postictal and postanesthetic confusion that lasts about 30 minutes, although the duration can (rarely) extend to hours. During this time, some patients (roughly 5 percent) may become severely agitated and require restraint and sedation. Preferred agents for this purpose include benzodiazepines, 1 to 2 mg intravenously, or diazepam, 5 to 10 mg intravenously) or antipsychotic medications. Factors that contribute to postictal confusion include frequency and number of ECT treatments, electrical dose, anesthetic agents used, and concomitant medication, including anticholinergic drugs and other CNS-active agents.

Memory loss, the major adverse effect of ECT, has both retrograde and anterograde components. Because of the repeated treatment, memory is characteristically worse for events occurring during the ECT course (anterograde amnesia). Most patients also experience retrograde amnesia that is usually worse for events occurring in the weeks prior to treatment. Typically, severity and duration of amnesia diminish as ECT administration becomes more remote. Some patients report difficulties with memory for more-distant events, including specific problems with autobiographical memories. These problems are often confounded by the fact that memory can be impaired by episodes of depression and other treatments used for depression. ECT-induced memory problems usually improve within 6 to 8 weeks following a course of treatment and coincide with the period during which the EEG shows significant slowing. Some patients report more-sustained difficulties with memory, lasting months, but persistent problems with memory formation are often difficult to demonstrate systematically, and interpretation can be confounded by recurrence of psychiatric symptoms.

As with postictal confusion, several variables contribute to memory impairment, including frequency and number of treatments, electrical charge used to induce seizures, and perhaps the drugs used for anesthesia. Electrode placement is possibly the greatest contributor of ECT-induced memory problems. Systematic studies clearly demonstrate that bilateral treatments are associated with significantly greater verbal memory impairment than nondominant hemisphere unilateral treatments. For this reason alone, unilateral electrode placement is considered the treatment of first choice for most patients referred for ECT.

Most of the major innovations in ECT technique, including the use of brief-pulse generators, titrated electrical doses, and nondominant hemisphere stimulation, have been directed toward minimizing this adverse effect while maintaining treatment efficacy Currently, it is believed that the therapeutic and adverse effects of ECT result from changes in CNS biochemistry and physiology. Furthermore, the beneficial effects of ECT require several treatments over a period of several days, which has spurred considerable interest in understanding the effects of repeated brief seizures on CNS functions. Even though there is no evidence that ECT produces structural damage to the brain, it is clear from the above disclosure that ECT alone usually has cognitive adverse effects, which would be significantly reduced by combining ECT with pulsed electrical stimulation to vagal nerve(s).

Advantageously, not only would the cognitive adverse effects be reduced, but the efficacy would also be significantly improved by the combination of ECT and VNS as disclosed in this application.

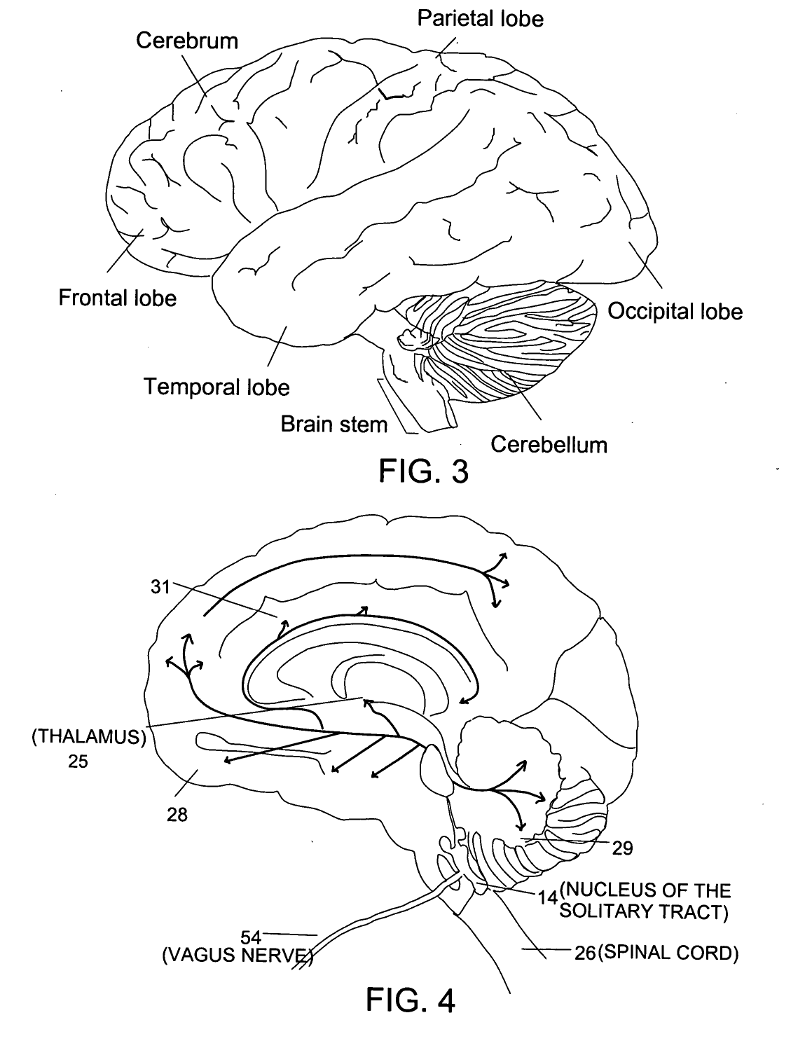

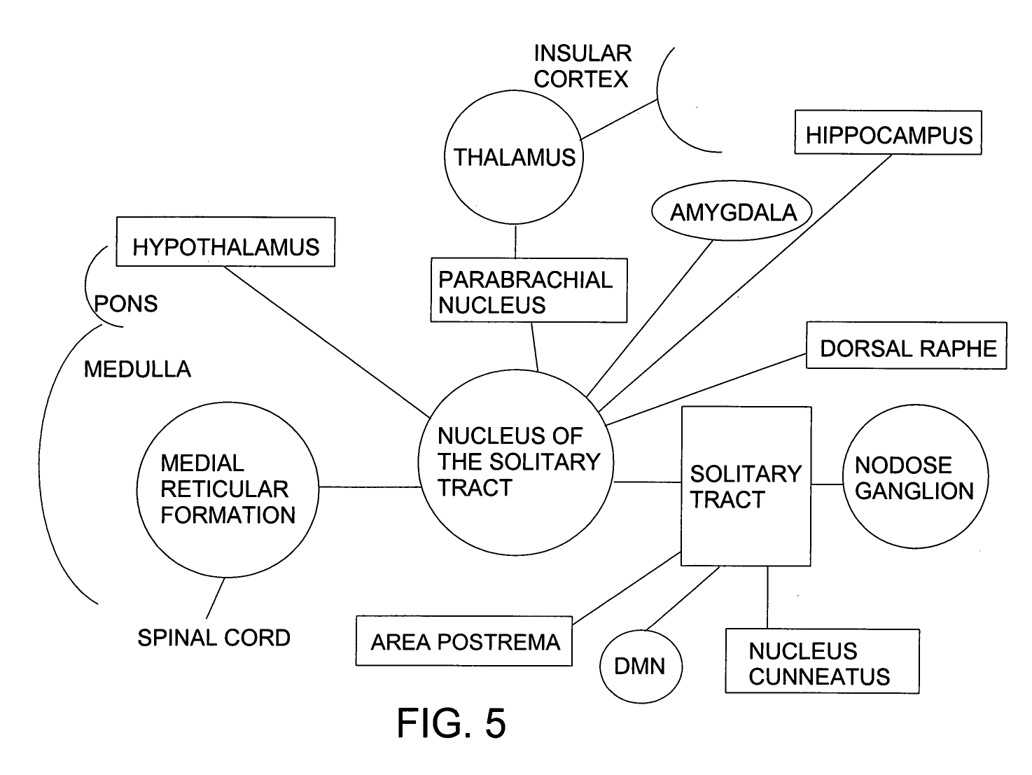

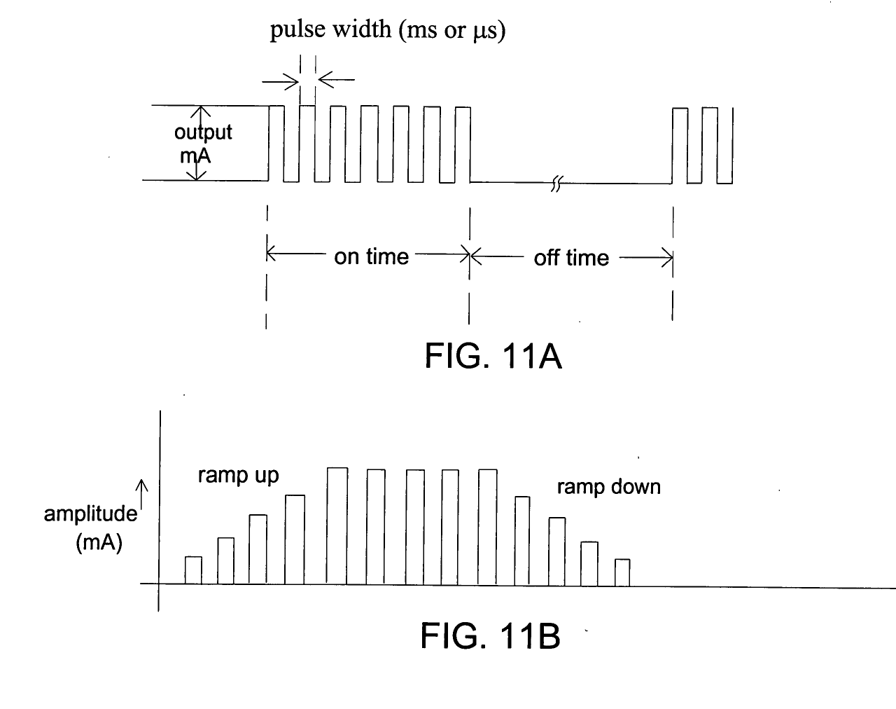

Therefore, in one aspect of the invention, modulation of some autonomic centers pertinent to the psychiatric disorders, is performed by providing pulsed electrical stimulation to vagus nerve(s) 54, which is shown in FIG. 4, and which is the X^{th} cranial nerve in the body. Other cranial nerves such as trigeminal nerve, or glossopharangeal nerve could also be used for this purpose. Since vagus nerve(s) is the easiest to expose, especially at the level of the neck, it is the preferred cranial nerve. Representative pulses provided to vagus nerve(s) are shown in conjunction with FIGS. 11A and 11B. Blocking pulses to selected branches may also be provided as disclosed later.

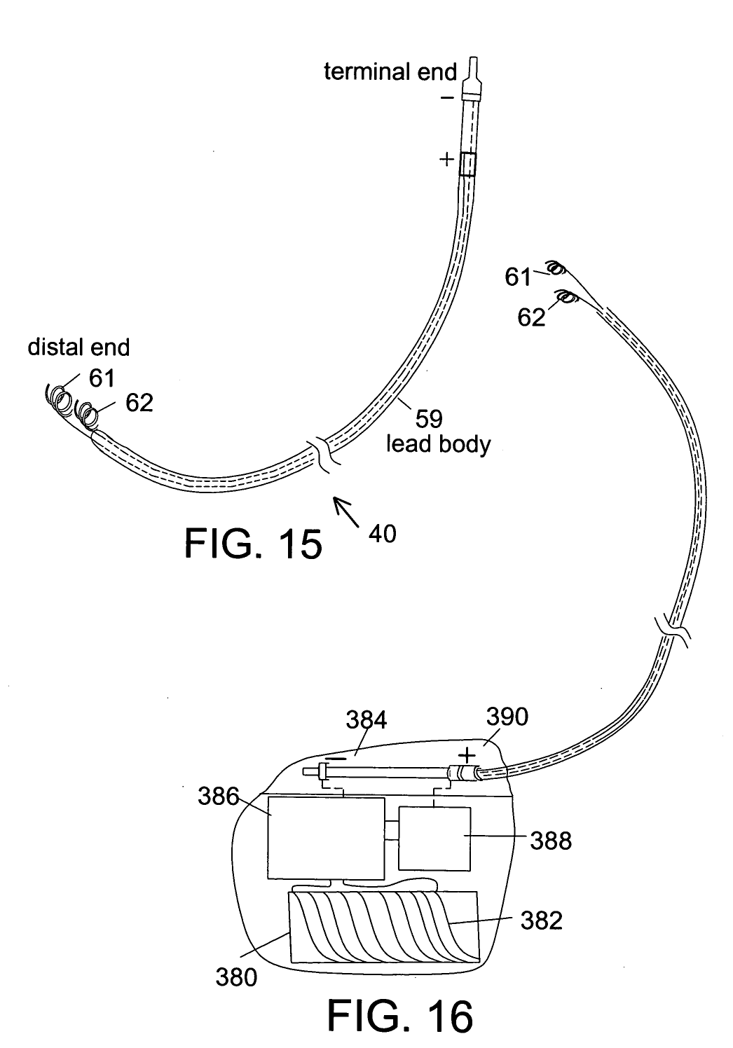

As was shown in conjunction with FIG. 1, pulsed electrical stimulation to the vagus nerve(s) 54 is provided utilizing a pulse generator means and an implanted lead 40. The implanted lead comprises a pair of electrodes 61, 62 (FIG. 15) that are adapted to be in contact with the vagus nerve(s) 54 for directly stimulating the nerve tissue. These electrodes may be placed on the vagus nerve 54 at around the neck level or around the diaphragmatic level, either just above or below the diaphragm. Also the electrodes may be implanted on one nerve for unilateral stimulation, or on both nerves for bilateral stimulation. The terminal end of the lead connects to either a pulse generator or a stimulus-receiver means.

Electrical pulses are provided to the vagus nerve(s) 54 using a system that comprises both implantable and external components. The system to provide selective stimulation (neuromodulation) may be selected from one of the following:
a) an implanted stimulus-receiver with an external stimulator;
b) an implanted stimulus-receiver comprising a high value capacitor for storing charge, used in conjunction with an external stimulator;
c) a programmer-less implantable pulse generator (IPG) which is operable with a magnet;
d) a microstimulator;
e) a programmable implantable pulse generator (IPG);
f) a combination implantable device comprising both a stimulus-receiver and a programmable IPG; and
g) an IPG comprising a rechargeable battery.

The pulse generator means is in electrical contact with a lead, which is adapted to be in contact with the vagus nerve(s) or its branches via electrodes. The pulse generator/stimulator can be of any form or type including those that are in current use, or in development, or to be developed in future. U.S. Pat. Nos. 4,702,254, 5,025,807, and 5,154,172 (Zabara) describe pulse generator and associated software to provide VNS therapy which are also included herein by reference, in this invention for application of VNS.

Using any of these systems, selective pulsed electrical stimulation is applied to vagus nerve(s) for afferent neuromodulation, at any point along the length of the nerve. The waveform of electrical pulses is shown in FIG. 11A. As shown in FIG. 11B, for patient comfort when the electrical stimulation is turned on, the electrical stimulation is ramped up and ramped down, instead of abrupt delivery of electrical pulses.

These stimulation systems for vagus nerve modulation are more fully described in a co-pending application (Ser. No. 10/841,995), but are mentioned here briefly for convenience. In each case, an implantable lead is surgically implanted in the patient 32. The vagus nerve(s) is/are surgically exposed and isolated. The electrodes on the distal end of the lead 40 are wrapped around the vagus nerve(s) 54, and the lead 40 is tunneled subcutaneously. A pulse generator means is connected to the proximal end of the lead. The power source may be external, implantable, or a combination device.

### Implanted Stimulus-Receiver with an External Stimulator

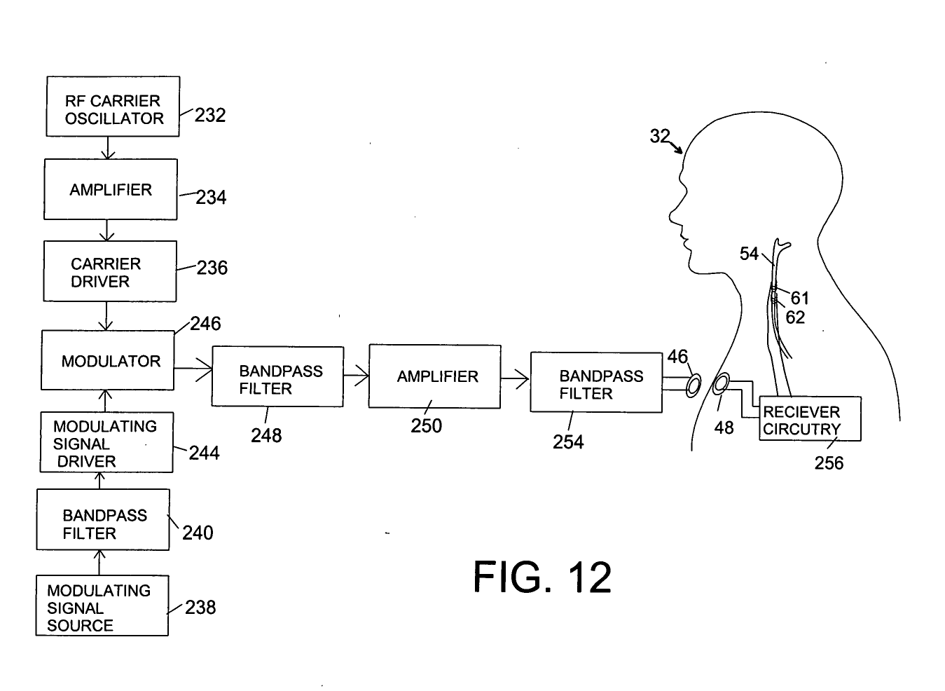

For utilizing an external power source, a passive implanted stimulus-receiver may be used. This embodiment of the vagus nerve pulse generator means is shown in conjunction with FIG. 12. A modulator 246 receives analog (sine wave) high frequency “carrier” signal and modulating signal. The modulating signal can be multilevel digital, binary, or even an analog signal. In this embodiment, mostly multilevel digital type modulating signals are used. The pulse amplitude and pulse width modulated signal is amplified 250, conditioned 254, and transmitted via a primary coil 46 which is external to the body. A secondary coil 48 of an implanted stimulus receiver, receives, demodulates, and delivers these pulses to the vagus nerve(s) 54 via electrodes 61 and 62. The receiver circuitry 256 is described later.

The carrier frequency is optimized. One preferred embodiment utilizes electrical signals of around 1 Mega-Hertz, even though other frequencies can be used. Low frequencies are generally not suitable because of energy requirements for longer wavelengths, whereas higher frequencies are absorbed by the tissues and are converted to heat, which again results in power losses.

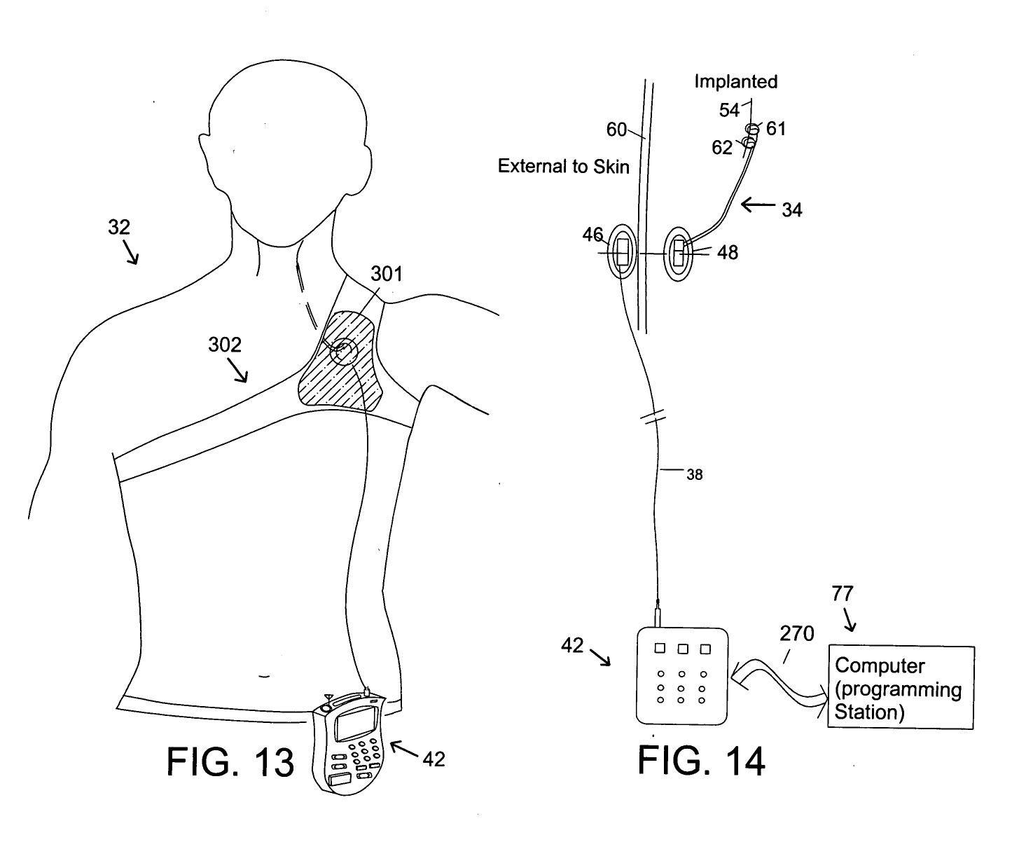

Shown in conjunction with FIG. 13, the coil for the external transmitter (primary coil 46) may be placed in the pocket 301 of a customized garment 302, for patient convenience.

Shown in conjunction with FIG. 14, the primary (external) coil 46 of the external stimulator 42 is inductively coupled to the secondary (implanted) coil 48 of the implanted stimulus-receiver 34. The implantable stimulus-receiver 34 has circuitry at the proximal end, and has two stimulating electrodes at the distal end 61,62. The negative electrode (cathode) 61 is positioned towards the brain and the positive electrode (anode) 62 is positioned away from the brain.

For therapy to commence, the primary (external) coil 46 is placed on the skin 60 on top of the surgically implanted (secondary) coil 48. An adhesive tape may be placed on the skin 60 and external coil 46 such that the external coil 46, is taped to the skin 60. For efficient energy transfer to occur, it is important that the primary (external) 46 and secondary (internal) coils 48 be positioned along the same axis and be optimally positioned relative to each other. In this embodiment, the external coil 46 may be connected to proximity sensing circuitry 50, in which case the correct positioning of the external coil 46 with respect to the internal coil 48 is indicated by turning “on” of a light emitting diode (LED) on the external stimulator 42.

The programmable parameters are stored in a programmable logic in the external stimulator 42. The predetermined programs stored in the external stimulator 42 are capable of being modified through the use of a separate programming station 77. A Programmable Array Logic Unit and interface unit are interfaced to the programming station 77. The programming station 77 can be used to load new programs, change the existing predetermined programs or the program parameters for various stimulation programs. The programming station is connected to the programmable array unit (comprising programmable array logic and interface unit) with an RS232-C serial connection. The main purpose of the serial line interface is to provide an RS232-C standard interface. Other suitable well known interface connections may also be used.

This method enables any portable computer with a serial interface to communicate and program the parameters for storing the various programs. The serial communication interface receives the serial data, buffers this data and converts it to a 16 bit parallel data. The programmable array logic component of programmable array unit (not shown) receives the parallel data bus and stores or modifies the data into a random access matrix. This array of data also contains special logic and instructions along with the actual data. These special instructions also provide an algorithm for storing, updating and retrieving the parameters from long-term memory. The programmable logic array unit, interfaces with long term memory to store the predetermined programs. All the previously modified programs can be stored here for access at any time, as well as, additional programs can be locked out for the patient. The programs consist of specific parameters and each unique program will be stored sequentially in long-term memory. A battery unit is present to provide power to all the components. The logic for the storage and decoding is stored in a random addressable storage matrix (RASM).

Conventional microprocessor and integrated circuits are used for the logic, control and timing circuits. Conventional bipolar transistors are used in radio-frequency oscillator, pulse amplitude ramp control and power amplifier. A standard voltage regulator is used in low-voltage detector. The hardware and software to deliver the pre-determined programs is well known to those skilled in the art.

The selective stimulation of the vagus nerve(s) can be performed in one of two ways. One method is to activate one of several “pre-determined/pre-packaged” programs. A second method is to “custom” program the electrical parameters, which can be selectively programmed for specific therapy to the individual patient. The electrical parameters that can be individually programmed, include variables such as pulse amplitude, pulse width, frequency of stimulation, stimulation on-time, and stimulation off-time. Table one below defines the approximate range of parameters,

TABLE 1
Electrical parameter range delivered to the nerve

| PARAMER | RANGE |
|---|---|
| Pulse Amplitude | 0.1 Volt-15 Volts |
| Pulse width | 20 μS-5 mSec. |
| Frequency | 5 Hz-200 Hz |
| On-time | 10 Secs-24 hours |
| Off-time | 10 Secs-24 hours |

The parameters in Table 1 are the electrical signals delivered to the nerve via the two electrodes 61,62 (distal and proximal) around the nerve, as shown in FIG. 14. It being understood that the signals generated by the external pulse generator 42 and transmitted via the primary coil 46 are larger, because the attenuation factor between the primary coil 46 and secondary coil 48 is approximately 10-20 times, depending upon coupling factors such as the distance, and orientation between the two coils. Accordingly, the range of transmitted signals of the external stimulator 42 may be approximately 10-20 times larger than shown in Table 1.

Referring to FIG. 15, the implanted lead component of the system is similar to cardiac pacemaker leads, except for distal portion (or electrode end) of the lead 40. The lead terminal preferably is linear bipolar, even though it can be bifurcated, and plug(s) into the cavity of the pulse generator means. The lead body 59 insulation may be constructed of medical grade silicone, silicone reinforced with polytetrafluoro-ethylene (PTFE), or polyurethane. The electrodes 61,62 for stimulating the vagus nerve 54 may either wrap around the nerve once or may be spiral shaped. These stimulating electrodes may be made of pure platinum, platinum/Iridium alloy or platinum/iridium coated with titanium nitride. The conductor connecting the terminal to the electrodes 61,62 is made of an alloy of nickel-cobalt. The implanted lead design variables are also summarized in table two below.

TABLE 2
Lead design variables

| | | | Conductor | | |
|---|---|---|---|---|---|
| Proximal | | | (connecting | | Distal |
| End | Lead body- | | proximal | | End |
| Lead | Insulation | | and distal | Electrode - | Electrode - |
| Terminal | Materials | Lead-Coating | ends) | Material | Type |
| Linear | Polyurethane | Antimicrobial | Alloy of | Pure | Spiral |
| bipolar | | coating | Nickel- | Platinum | electrode |
| | | | Cobalt | | |
| Bifurcated | Silicone | Anti- | | Platinum- | Wrap-around |
| | | Inflammatory | | Iridium | electrode |
| | | coating | | (Pt/Ir) Alloy | |
| | Silicone with | Lubricious | | Pt/Ir coated | Steroid |
| | Polytetrafluoro- | coating | | with Titanium | eluting |
| | ethylene | | | Nitride | |
| | (PTFE) | | | | |
| | | | | Carbon | Hydrogel |
| | | | | | electrodes |
| | | | | | Cuff |
| | | | | | electrodes |

Once the lead is fabricated, coating such as anti-microbial, anti-inflammatory, or lubricious coating may be applied to the lead body 59.

### Implanted Stimulus-Receiver Comprising a High Value Capacitor for Storing Charge, Used in Conjunction with an External Stimulator

In one embodiment, the implanted stimulus-receiver may be a system which is RF coupled combined with a power source. In this embodiment, the implanted stimulus-receiver comprises high value, small sized capacitor(s) for storing charge and delivering electric stimulation pulses for up to several hours by itself, once the capacitors are charged. The packaging is shown in FIG. 16. Using mostly hybrid components and appropriate packaging, the implanted portion of the system described below can be miniaturized. As shown in FIG. 16, a solenoid coil 382 wrapped around a ferrite core 380 is used as the secondary of an air-gap transformer for receiving power and data to the implanted device. The primary coil is external to the body. Since the coupling between the external transmitter coil and receiver coil 382 may be weak, a high-efficiency transmitter/amplifier is used in order to supply enough power to the receiver coil 382. Class-D or Class-E power amplifiers may be used for this purpose. The coil for the external transmitter (primary coil) may be placed in the pocket of a customized garment, as was shown previously in FIG. 13.

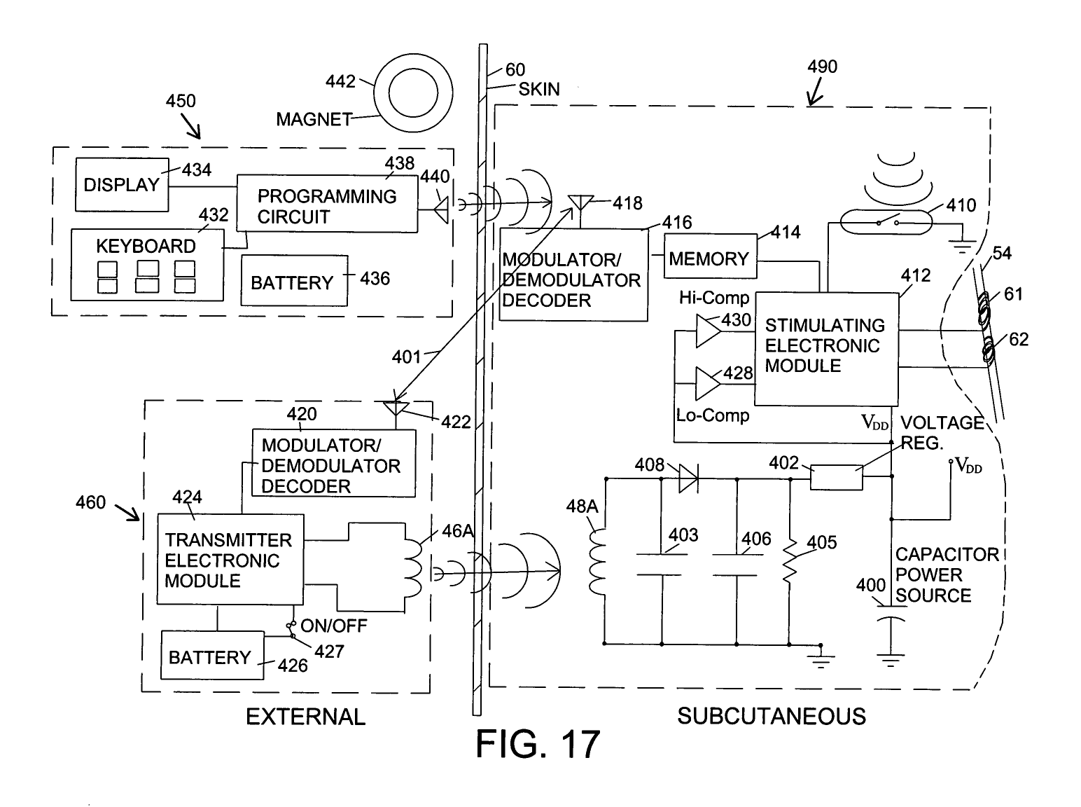

Shown in conjunction with FIG. 17 of the implanted stimulus-receiver 490 and the system, the receiving inductor 48A and tuning capacitor 403 are tuned to the frequency of the transmitter. The diode 408 rectifies the AC signals, and a small sized capacitor 406 is utilized for smoothing the input voltage V_{I} fed into the voltage regulator 402. The output voltage V_{D} of regulator 402 is applied to capacitive energy power supply and source 400 which establishes source power V_{DD}. Capacitor 400 is a big value, small sized capacative energy source which is classified as low internal impedance, low power loss and high charge rate capacitor, such as Panasonic Model No. 641.

The refresh-recharge transmitter unit 460 includes a primary battery 426, an ON/Off switch 427, a transmitter electronic module 424, an RF inductor power coil 46A, a modulator/demodulator 420 and an antenna 422.

When the ON/OFF switch is on, the primary coil 46A is placed in close proximity to skin 60 and secondary coil 48A of the implanted stimulator 490. The inductor coil 46A emits RF waves establishing EMF wave fronts which are received by secondary inductor 48A. Further, transmitter electronic module 424 sends out command signals which are converted by modulator/demodulator decoder 420 and sent via antenna 422 to antenna 418 in the implanted stimulator 490. These received command signals are demodulated by decoder 416 and replied and responded to, based on a program in memory 414 (matched against a “command table” in the memory). Memory 414 then activates the proper controls and the inductor receiver coil 48A accepts the RF coupled power from inductor 46A.

The RF coupled power, which is alternating or AC in nature, is converted by the rectifier 408 into a high DC voltage. Small value capacitor 406 operates to filter and level this high DC voltage at a certain level. Voltage regulator 402 converts the high DC voltage to a lower precise DC voltage while capacitive power source 400 refreshes and replenishes.

When the voltage in capacative source 400 reaches a predetermined level (that is V_{DD} reaches a certain predetermined high level), the high threshold comparator 430 fires and stimulating electronic module 412 sends an appropriate command signal to modulator/decoder 416. Modulator/decoder 416 then sends an appropriate “fully charged” signal indicating that capacitive power source 400 is fully charged, is received by antenna 422 in the refresh-recharge transmitter unit 460.

In one mode of operation, the patient may start or stop stimulation by waving the magnet 442 once near the implant. The magnet emits a magnetic force Lₘ which pulls reed switch 410 closed. Upon closure of reed switch 410, stimulating electronic module 412 in conjunction with memory 414 begins the delivery (or cessation as the case may be) of controlled electronic stimulation pulses to the vagus nerve(s) 54 via electrodes 61, 62. In another mode (AUTO), the stimulation is automatically delivered to the implanted lead based upon programmed ON/OFF times.

The programmer unit 450 includes keyboard 432, programming circuit 438, rechargeable battery 436, and display 434. The physician or medical technician programs programming unit 450 via keyboard 432. This program regarding the frequency, pulse width, modulation program, ON time etc. is stored in programming circuit 438. The programming unit 450 must be placed relatively close to the implanted stimulator 490 in order to transfer the commands and programming information from antenna 440 to antenna 418. Upon receipt of this programming data, modulator/demodulator and decoder 416 decodes and conditions these signals, and the digital programming information is captured by memory 414. This digital programming information is further processed by stimulating electronic module 412. In the DEMAND operating mode, after programming the implanted stimulator, the patient turns ON and OFF the implanted stimulator via hand held magnet 442 and the reed switch 410. In the automatic mode (AUTO), the implanted stimulator turns ON and OFF automatically according to the programmed values for the ON and OFF times.

Other simplified versions of such a system may also be used. For example, a system such as this, where a separate programmer is eliminated, and simplified programming is performed with a magnet and reed switch, can also be used.

### Programmer-Less Implantable Pulse Generator (IPG)

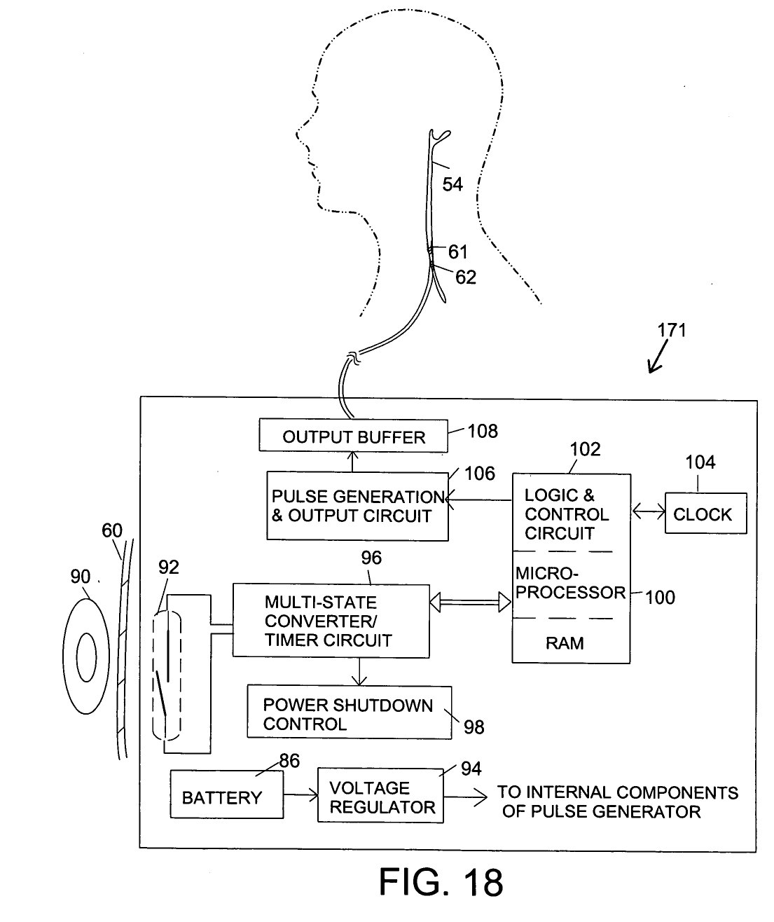

In one embodiment, a programmer-less implantable pulse generator (IPG) may be used. In this embodiment, shown in conjunction with FIG. 18, the implantable pulse generator 171 is provided with a reed switch 92 and memory & control circuitry 102. The reed switch 92 being remotely actuable by means of a magnet 90 brought into proximity of the pulse generator 171, in accordance with common practice in the art. In this embodiment, the reed switch 92 is coupled to a multi-state converter/timer circuit 96, such that a single short closure of the reed switch can be used as a means for non-invasive encoding and programming of the pulse generator 171 parameters.

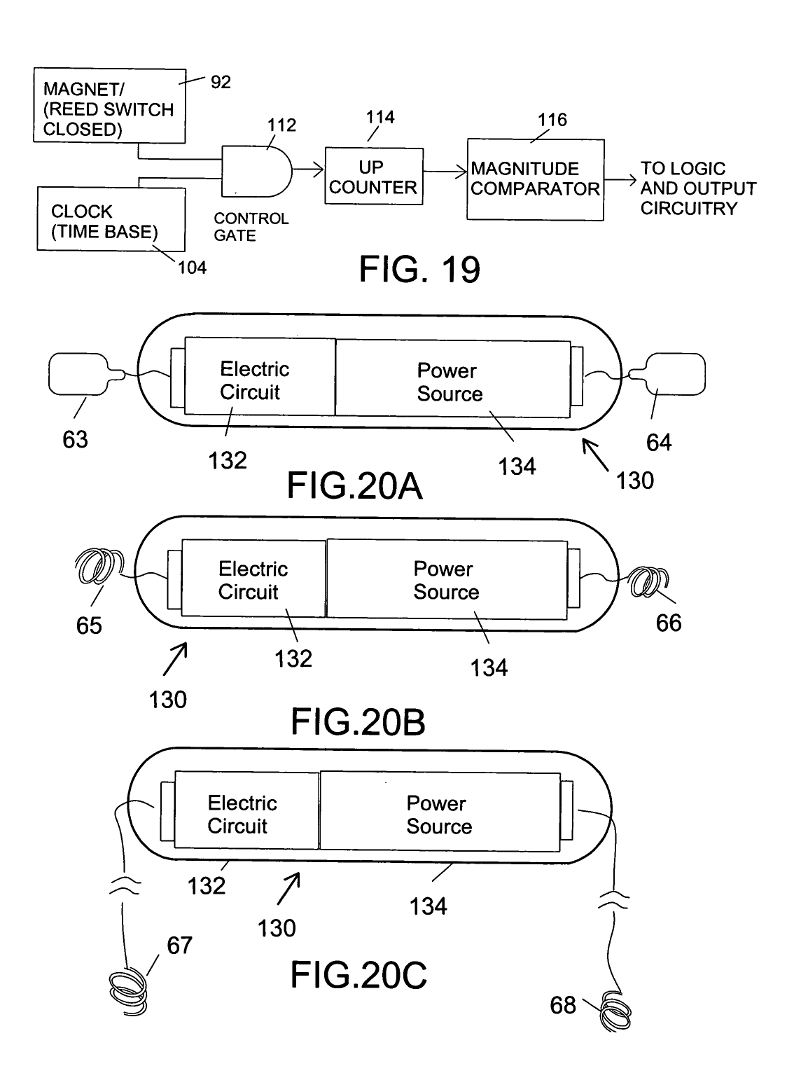

In one embodiment, shown in conjunction with FIG. 19, the closing of the reed switch 92 triggers a counter. The magnet 90 and timer are ANDed together. The system is configured such that during the time that the magnet 82 is held over the pulse generator 171, the output level goes from LOW stimulation state to the next higher stimulation state every 5 seconds. Once the magnet 82 is removed, regardless of the state of stimulation, an application of the magnet, without holding it over the pulse generator 171, triggers the OFF state, which also resets the counter.

Once the prepackaged/predetermined logic state is activated by the logic and control circuit 102, the pulse generation and amplification circuit 106 deliver the appropriate electrical pulses to the vagus nerve(s) 54 of the patient via an output buffer 108 (as shown in FIG. 18). The delivery of output pulses is configured such that the distal electrode 61 (electrode closer to the brain) is the cathode, and the proximal electrode 62 is the anode. Timing signals for the logic and control circuit 102 of the pulse generator 171 are provided by a crystal oscillator 104. The battery 86 of the pulse generator 171 has terminals connected to the input of a voltage regulator 94. The regulator 94 smoothes the battery output and supplies power to the internal components of the pulse generator 171. A microprocessor 100 controls the program parameters of the device, such as the voltage, pulse width, frequency of pulses, on-time and off-time. The microprocessor 100 may be a commercially available, general purpose microprocessor or microcontroller, or may be a custom integrated circuit device augmented by standard RAM/ROM components.

In one embodiment, there are four stimulation states. A larger (or lower) number of states can be achieved using the same methodology, and such is considered within the scope of the invention. These four states are, LOW stimulation state, LOW-MED stimulation state, MED stimulation state, and HIGH stimulation state. Examples of stimulation parameters (delivered to the vagus nerve) for each state are as follows,

LOW stimulation state example is,

| | |
|---|---|
| Current output: | 0.75 milliAmps. |
| Pulse width: | 0.20 msec. |
| Pulse frequency: | 20 Hz |
| Cycles: | 20 sec. on-time and 2.0 min. off-time |
| | in repeating cycles. |

LOW-MED stimulation state example is,

| | |
|---|---|
| Current output: | 1.5 milliAmps, |
| Pulse width: | 0.30 msec. |
| Pulse frequency: | 25 Hz |
| Cycles: | 1.5 min. on-time and 20.0 min. off-time |
| | in repeating cycles. |

MED stimulation state example is,

| | |
|---|---|
| Current output: | 2.0 milliAmps. |
| Pulse width: | 0.30 msec. |
| Pulse frequency: | 30 Hz |
| Cycles: | 1.5 min. on-time and 20.0 min. off-time |
| | in repeating cycles. |

HIGH stimulation state example is,

| | |
|---|---|
| Current output: | 3.0 milliAmps, |
| Pulse width: | 0.40 msec. |
| Pulse frequency: | 30 Hz |
| Cycles: | 2.0 min. on-time and 20.0 min. off-time |
| | in repeating cycles. |

These prepackaged/predetermined programs are mearly examples, and the actual stimulation parameters will deviate from these depending on the patient or treatment application.

It will be readily apparent to one skilled in the art, that other schemes can be used for the same purpose. For example, instead of placing the magnet 90 on the pulse generator 171 for a prolonged period of time, different stimulation states can be encoded by the sequence of magnet applications. Accordingly, in an alternative embodiment there can be three logic states, OFF, LOW stimulation (LS) state, and HIGH stimulation (HS) state. Each logic state again corresponds to a prepackaged/predetermined program such as presented above. In such an embodiment, the system could be configured such that one application of the magnet 90 triggers the generator into LS State. If the generator is already in the LS state then one application triggers the device into OFF State. Two successive magnet applications triggers the generator into MED stimulation state, and three successive magnet applications triggers the pulse generator in the HIGH Stimulation State. Subsequently, one application of the magnet while the device is in any stimulation state, turns the device OFF.

The advantage of this embodiment is that it is cheaper to manufacture than a fully programmable implantable pulse generator (IPG).

### Microstimulator

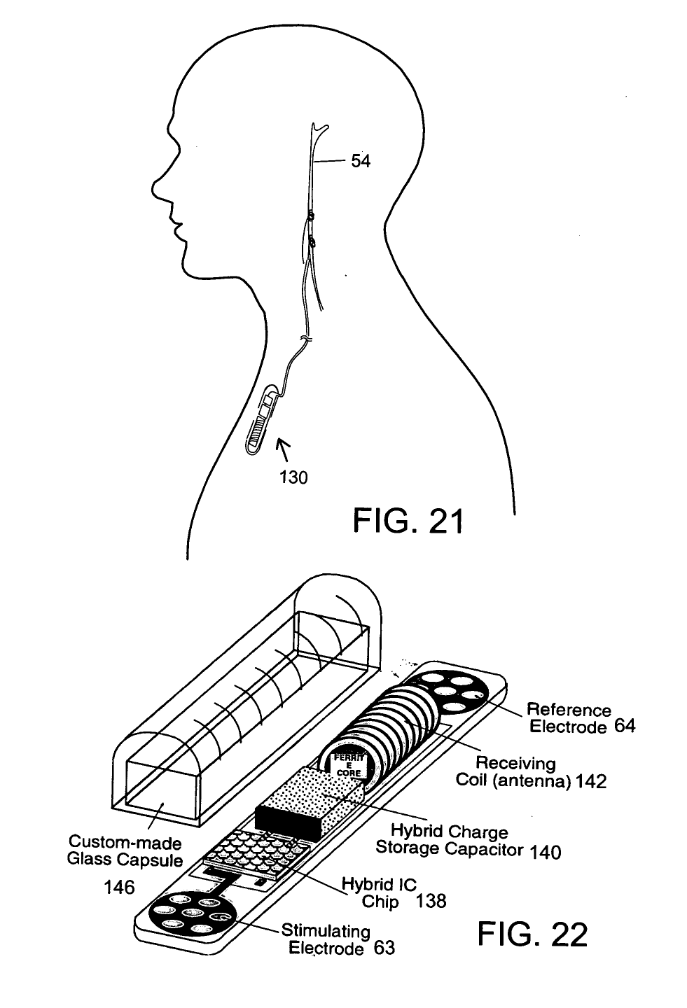

In one embodiment, a microstimulator 130 may be used for providing pulses to the vagus nerve(s) 54. Shown in conjunction with FIG. 20A, is a microstimulator where the electrical circuitry 132 and power source 134 are encased in a miniature hermetically sealed enclosure, and only the electrodes 63, 64 are exposed. FIG. 20B depicts the same microstimulator, except the electrodes are modified and adapted to wrap around the nerve tissue 54. Because of its small size, the whole microstimulator may be in the proximity of the nerve tissue to be stimulated, or alternatively as shown in conjunction with FIG. 21, the microstimulator may be implanted at a different site, and connected to the electrodes via conductors insulated with silicone and polyurethane (FIG. 20C).

Shown in reference with FIG. 22 is the overall structure of an implantable microstimulator 130. It consists of a micromachined silicon substrate that incorporates two stimulating electrodes which are the cathode and anode of a bipolar stimulating electrode pair 63, 64; a hybrid-connected tantalum chip capacitor 140 for power storage; a receiving coil 142; a bipolar-CMOS integrated circuit chip 138 for power regulation and control of the microstimulator; and a custom made glass capsule 146 that is electrostatically bonded to the silicon carrier to provide a hermetic package for the receiver-stimulator circuitry and hybrid elements. The stimulating electrode pair 63,64 resides outside of the package and feedthroughs are used to connect the internal electronics to the electrodes.

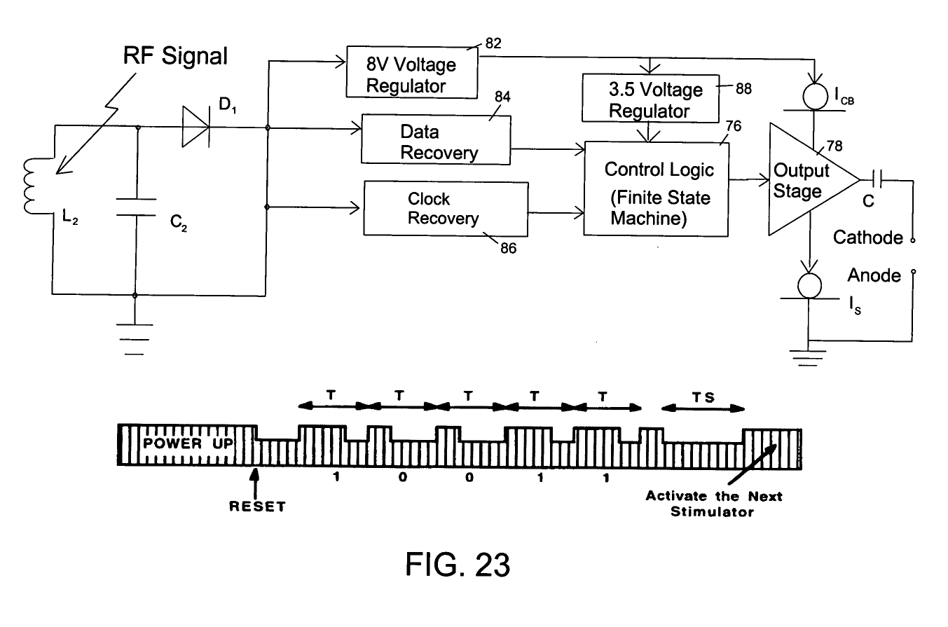

FIG. 23 shows the overall system electronics required for the microstimulator, and the power and data transmission protocol used for radiofrequency telemetry. The circuit receives an amplitude modulated RF carrier from an external transmitter and generates 8-V and 4-V dc supplies, generates a clock from the carrier signal, decodes the modulated control data, interprets the control data, and generates a constant current output pulse when appropriate. The RF carrier used for the telemetry link has a nominal frequency of around 1.8 MHz, and is amplitude modulated to encode control data. Logical “1” and “0” are encoded by varying the width of the amplitude modulated carrier, as shown in the bottom portion of FIG. 23. The carrier signal is initially high when the transmitter is turned on and sets up an electromagnetic field inside the transmitter coil. The energy in the field is picked up by receiver coils 142, and is used to charge the hybrid capacitor 140. The carrier signal is turned high and then back down again, and is maintained at the low level for a period between 1-200 μsec. The microstimulator 130 will then deliver a constant current pulse into the nerve tissue through the stimulating electrode pair 63, 64 for the period that the carrier is low. Finally, the carrier is turned back high again, which will indicate the end of the stimulation period to the microstimulator 130, thus allowing it to charge its capacitor 140 back up to the on-chip voltage supply.

On-chip circuitry has been designed to generate two regulated power supply voltages (4V and 8V) from the RF carrier, to demodulate the RF carrier in order to recover the control data that is used to program the microstimulator, to generate the clock used by the on-chip control circuitry, to deliver a constant current through a controlled current driver into the nerve tissue, and to control the operation of the overall circuitry using a low-power CMOS logic controller.

### Programmable Implantable Pulse Generator (IPG)

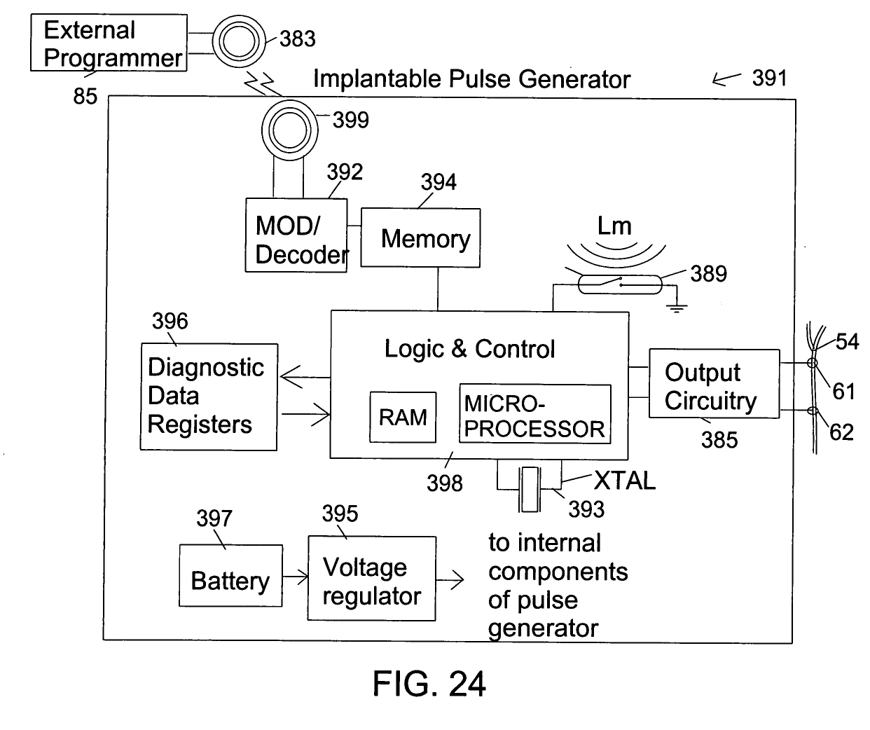

In one embodiment, a fully programmable implantable pulse generator (IPG) may be used. Shown in conjunction with FIG. 24, the implantable pulse generator unit 391 is preferably a microprocessor based device, where the entire circuitry is encased in a hermetically sealed titanium can. As shown in the overall block diagram, the logic & control unit 398 provides the proper timing for the output circuitry 385 to generate electrical pulses that are delivered to electrodes 61, 62 via a lead 40 (not shown). Programming of the implantable pulse generator (IPG) 391 is done via an external programmer 85. Once programmed via an external programmer 85, the implanted pulse generator 391 provides appropriate electrical stimulation pulses to the vagus nerve(s) 54 via electrodes 61,62.

This embodiment may also comprise optional fixed pre-determined/pre-packaged programs. Examples of LOW, LOW-MED, MED, and HIGH stimulation states were given in the previous section, under “Programmer-less Implantable Pulse Generator (IPG)”. These pre-packaged/pre-determined programs comprise unique combinations of pulse amplitude, pulse width, pulse frequency, ON-time and OFF-time. Advantageously, a number of these “pre-determined/pre-packaged programs” may be stored in a “library”, and activated in a simple fashion, without having to program each parameter individually.

In addition, each parameter may be individually programmed and stored in memory. The range of programmable electrical stimulation parameters are shown in table 3 below.

TABLE 3
Programmable electrical parameter range

| PARAMER | RANGE |
|---|---|
| Pulse Amplitude | 0.1 Volt-15 Volts |
| Pulse width | 20 μS-5 mSec. |
| Frequency | 3 Hz-300 Hz |
| On-time | 5 Secs-24 hours |
| Off-time | 5 Secs-24 hours |
| Ramp | ON/OFF |

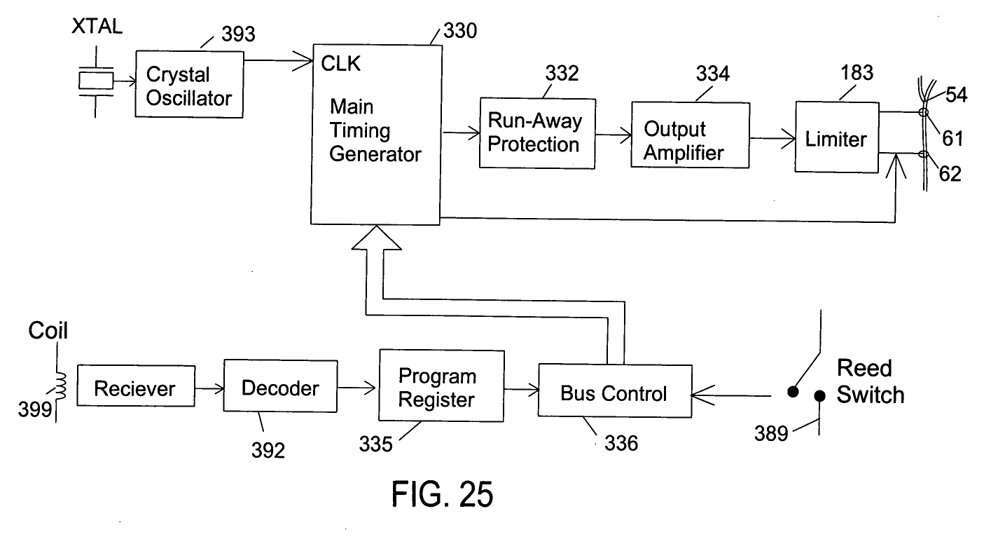

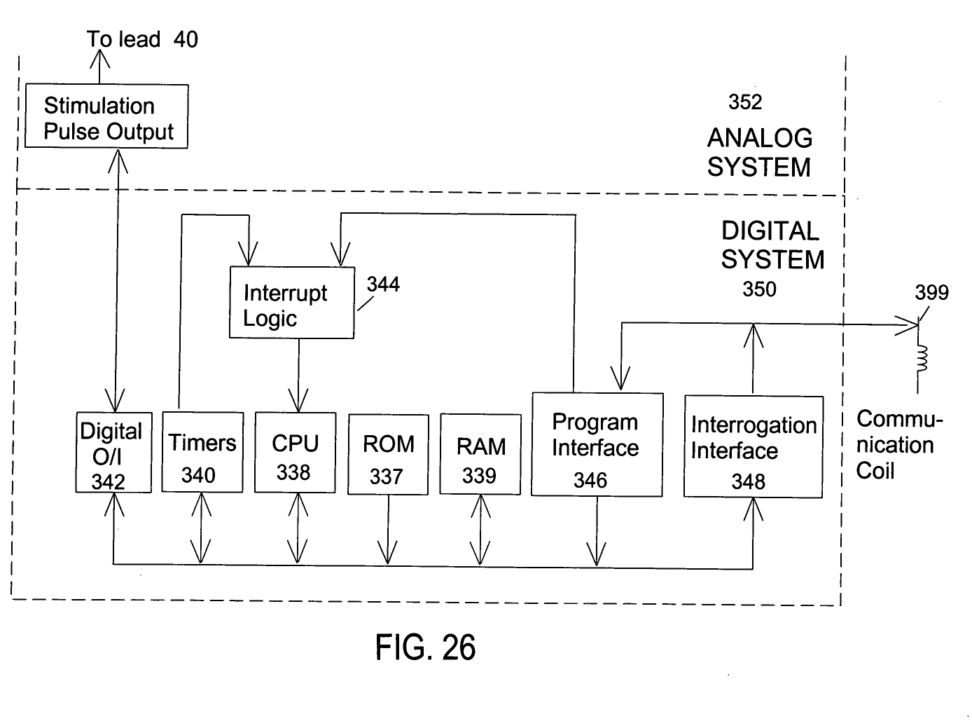

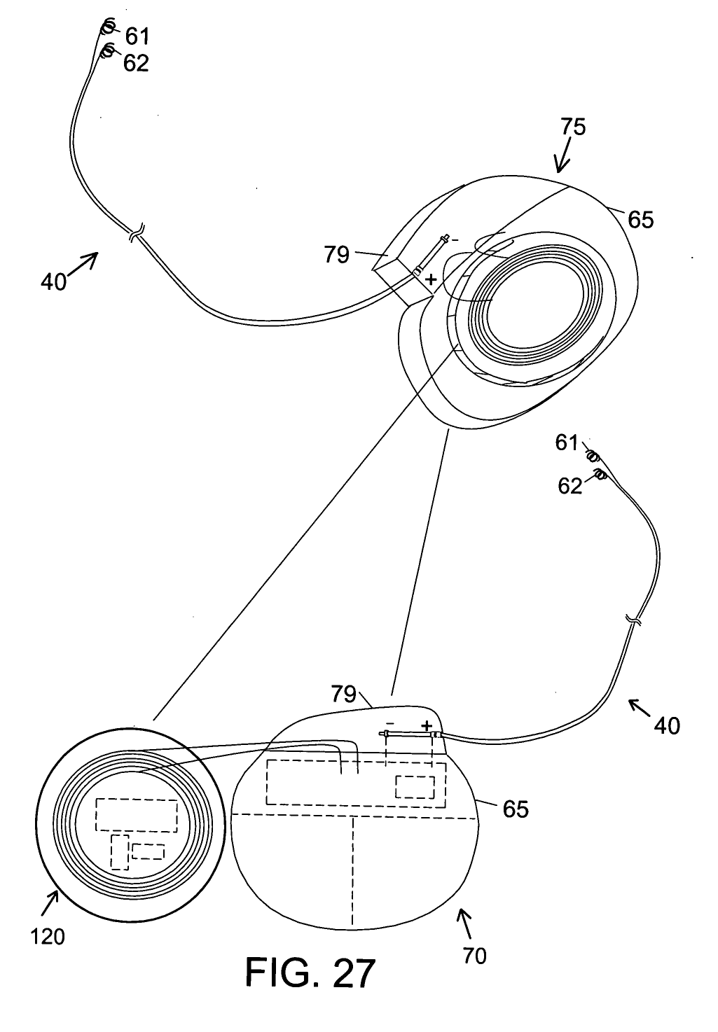

Shown in conjunction with FIGS. 25 and 26, the electronic stimulation module comprises both digital 350 and analog 352 circuits. A main timing generator 330 (shown in FIG. 25), controls the timing of the analog output circuitry for delivering neuromodulating pulses to the vagus nerve(s) 54, via output amplifier 334. Limiter 183 prevents excessive stimulation energy from getting into the vagus nerve(s) 54. The main timing generator 330 receiving clock pulses from crystal oscillator 393. Main timing generator 330 also receiving input from programmer 85 via coil 399. FIG. 26 highlights other portions of the digital system such as CPU 338, ROM 337, RAM 339, program interface 346, interrogation interface 348, timers 340, and digital O/I 342. The functioning details of these circuits is well known to one skilled in the art.

Most of the digital functional circuitry 350 is on a single chip (IC). This monolithic chip along with other IC's and components such as capacitors and the input protection diodes are assembled together on a hybrid circuit. As well known in the art, hybrid technology is used to establish the connections between the circuit and the other passive components. The integrated circuit is hermetically encapsulated in a chip carrier. A coil 399 connected to the hybrid is used for bidirectional telemetry. The hybrid and battery 397 are encased in a titanium can. This housing is a two-part titanium capsule that is hermetically sealed by laser welding. Alternatively, electron-beam welding can also be used. The header 79 is a cast epoxy-resin with hermetically sealed feed-through, and form the lead 40 connection block.

### Combination Implantable Device Comprising Both a Stimulus-Receiver and a Programmable Implantable Pulse Generator (IPG)

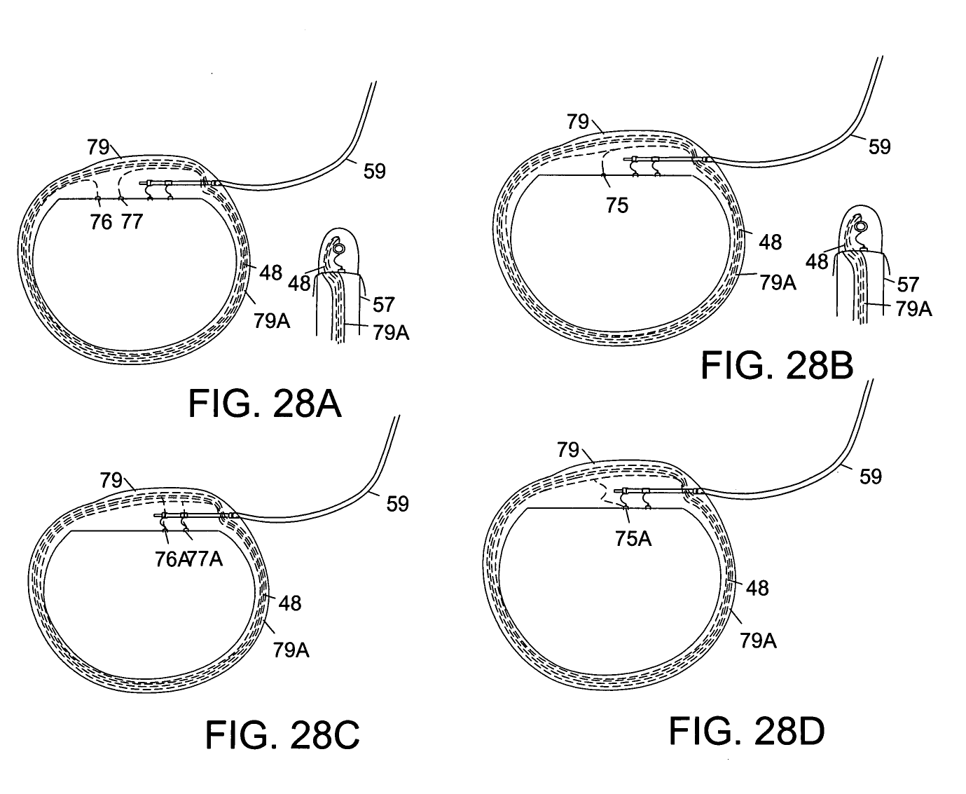

In one embodiment, the implantable device may comprise both a stimulus-receiver and a programmable implantable pulse generator (IPG) in one device. FIG. 27 shows a close up view of the packaging of the implanted stimulator 75 of this embodiment, showing the two subassemblies 120, 70. The two subassemblies are the stimulus-receiver module 120 and the battery operated pulse generator module 70. The electrical components of the stimulus-receiver module 120 may be substantially in the titanium case along with other circuitry, except for a coil. The coil may be outside the titanium case as shown in FIG. 27, or the coil 48C may be externalized at the header portion 79C of the implanted device, and may be wrapped around the titanium can. In this case, the coil is encased in the same material as the header 79C, as shown in FIGS. 28A-D. FIG. 28A depicts a bipolar configuration with two separate feed-throughs, 76, 77. FIG. 28B depicts a unipolar configuration with one separate feed-through. FIG. 28C, and 28D depict the same configuration except the feed-throughs are common with the feed-throughs for the lead.

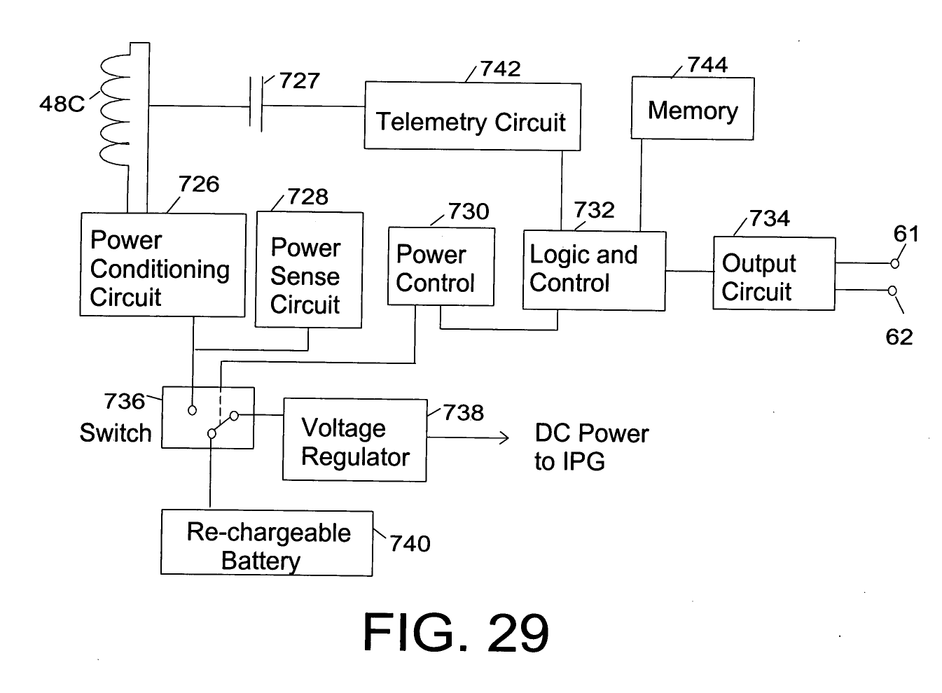

FIG. 29 is a simplified overall block diagram of the embodiment where the implanted stimulator 75 is a combination device, which may be used as a stimulus-receiver (SR) in conjunction with an external stimulator, or the same implanted device may be used as a traditional programmable implanted pulse generator (IPG). The coil 48C which is external to the titanium case may be used both as a secondary of a stimulus-receiver, or may also be used as the forward and back telemetry coil.

In this embodiment, as disclosed in FIG. 29, the IPG circuitry within the titanium case is used for all stimulation pulses whether the energy source is the internal battery 740 or an external power source. The external device serves as a source of energy, and as a programmer that sends telemetry to the IPG. For programming, the energy is sent as high frequency sine waves with superimposed telemetry wave driving the external coil 46C. Once received by the implanted coil 48C, the telemetry is passed through coupling capacitor 727 to the IPG's telemetry circuit 742. For pulse delivery using external power source, the stimulus-receiver portion will receive the energy coupled to the implanted coil 48C and, using the power conditioning circuit 726, rectify it to produce DC, filter and regulate the DC, and couple it to the IPG's voltage regulator 738 section so that the IPG can run from the externally supplied energy rather than the implanted battery 740.

The system provides a power sense circuit 728 that senses the presence of external power communicated with the power control 730 when adequate and stable power is available from an external source. The power control circuit controls a switch 736 that selects either battery power 740 or conditioned external power from 726. The logic and control section 732 and memory 744 includes the IPG's microcontroller, pre-programmed instructions, and stored chagneable parameters. Using input for the telemetry circuit 742 and power control 730, this section controls the output circuit 734 that generates the output pulses.

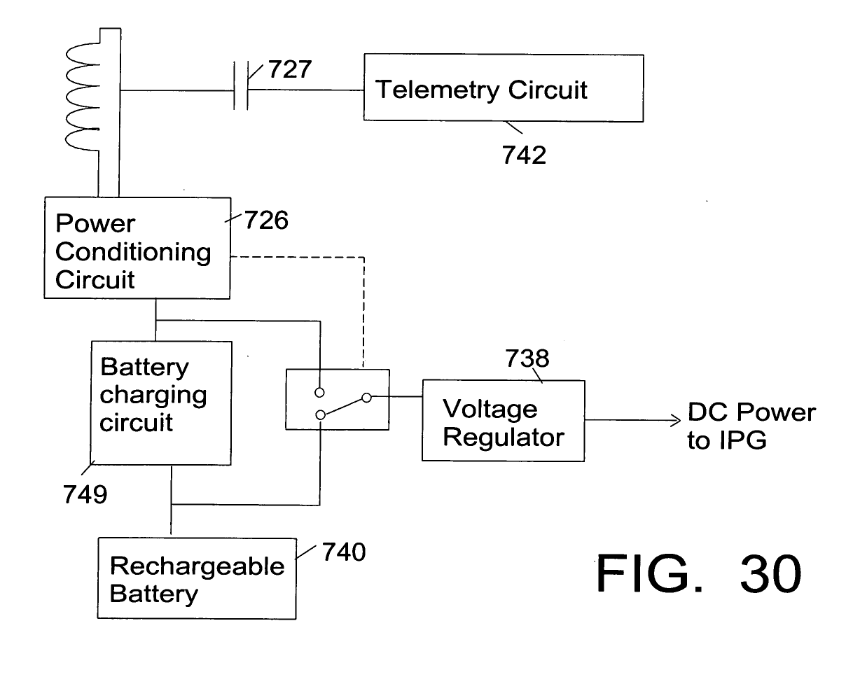

It will be clear to one skilled in the art that this embodiment of the invention can also be practiced with a rechargeable battery. This version is shown in conjunction with FIG. 30. The circuitry in the two versions are similar except for the battery charging circuitry 749. This circuit is energized when external power is available. It senses the charge state of the battery and provides appropriate charge current to safely recharge the battery without overcharging.

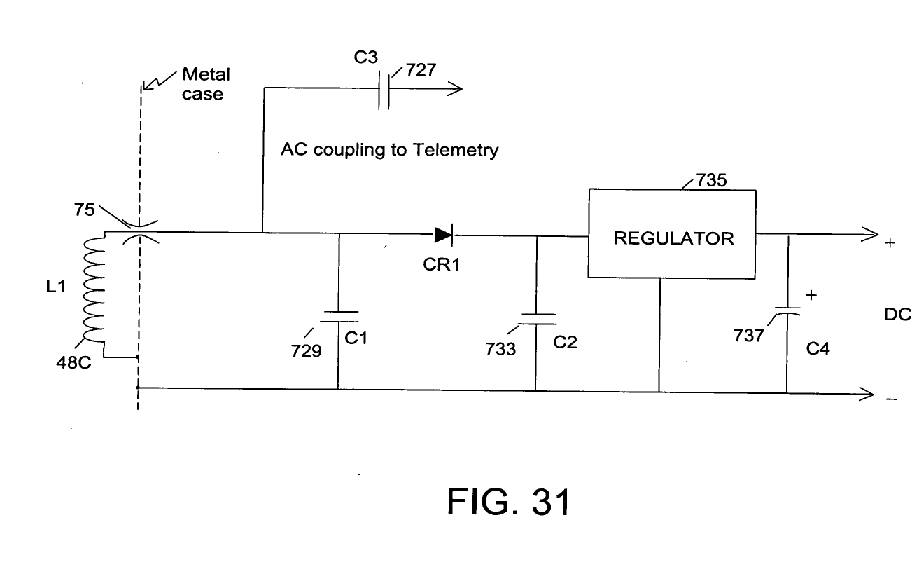

The stimulus-receiver portion of the circuitry is shown in conjunction with FIG. 31. Capacitor C1 (729) makes the combination of C1 and L1 sensitive to the resonant frequency and less sensitive to other frequencies, and energy from an external (primary) coil 46C is inductively transferred to the implanted unit via the secondary coil 48C. The AC signal is rectified to DC via diode 731, and filtered via capacitor 733. A regulator 735 sets the output voltage and limits it to a value just above the maximum IPG cell voltage. The output capacitor C4 (737), typically a tantalum capacitor with a value of 100 micro-Farads or greater, stores charge so that the circuit can supply the IPG with high values of current for a short time duration with minimal voltage change during a pulse while the current draw from the external source remains relatively constant. Also shown in conjunction with FIG. 31, a capacitor C3 (727) couples signals for forward and back telemetry.

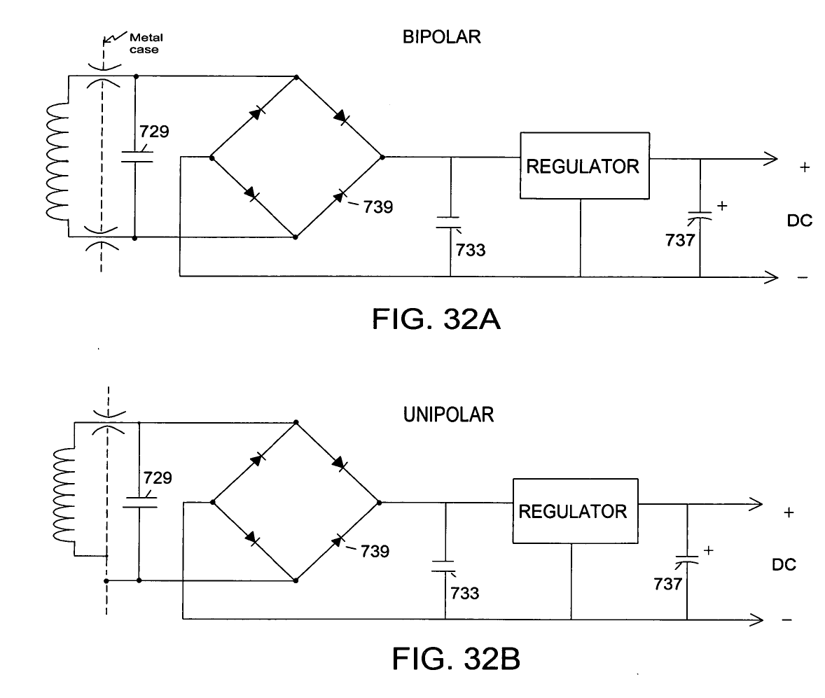

FIGS. 32A and 32B show alternate connection of the receiveing coil. In FIG. 32A, each end of the coil is connected to the circuit through a hermetic feedthrough filter. In this instance, the DC output is floating with respect to the IPG's case. In FIG. 32B, one end of the coil is connected to the exterior of the IPG's case. The circuit is completed by connecting the capacitor 729 and bridge rectifier 739 to the interior of the IPG's case The advantage of this arrangement is that it requires one less hermetic feedthrough filter, thus reducing the cost and improving the reliabilty of the IPG. Hermetic feedthrough filters are expensive and a possible failure point. However, the case connection may complicit the output circuitry or limit its versatility. When using a bipolar electrode, care must be taken to prevent an unwanted return path for the pulse to the IPG's case. This is not a concern for unipolar pulses using a single conductor electrode because it relies on the IPG's case a return for the pulse current.

In the unipolar configuration, advantageously a bigger tissue area is stimulated since the difference between the tip (cathode) and case (anode) is larger. Stimulation using both configuration is considered within the scope of this invention.

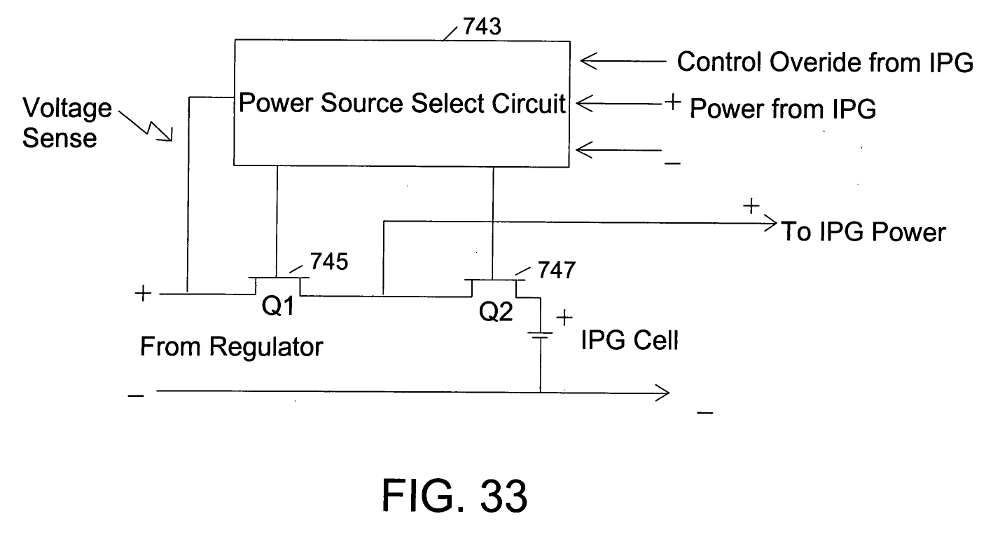

The power source select circuit is highlighted in conjunction with FIG. 33. In this embodiment, the IPG provides stimulation pulses according to the stimulation programs stored in the memory 744 of the implanted stimulator, with power being supplied by the implanted battery 740. When stimulation energy from an external stimulator is inductively received via secondary coil 48C, the power source select circuit (shown in block 743) switches power via transistor Q1 745 and transistor Q2 743. Transistor Q1 and Q2 are preferably low loss MOS transistor used as switches, even though other types of transistors may be used.

### Implantable Pulse Generator (IPG) Comprising a Rechargable Battery

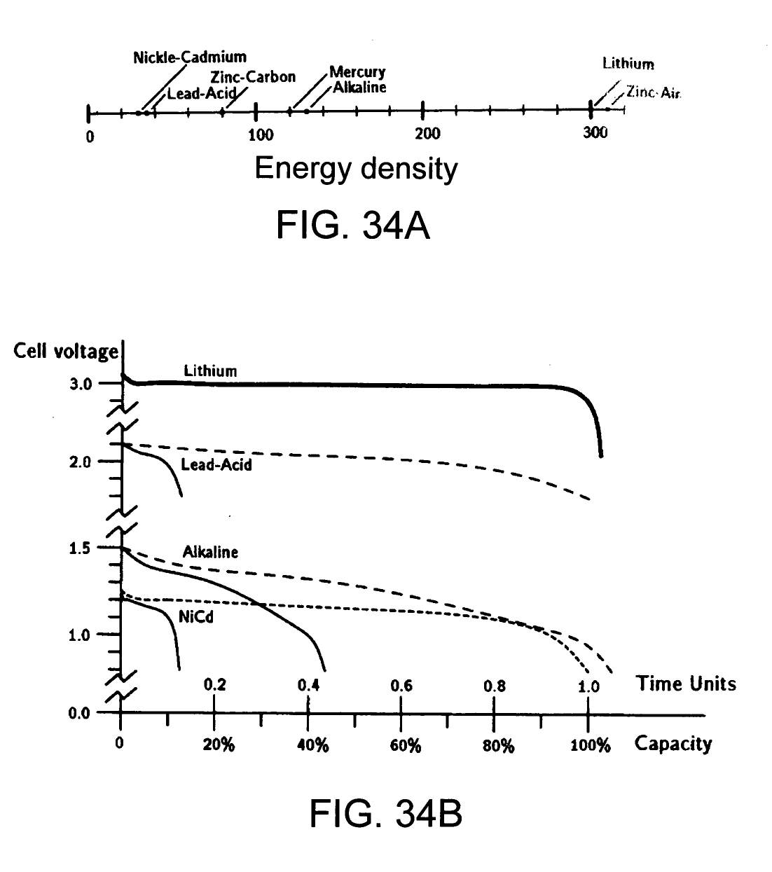

In one embodiment, an implantable pulse generator with rechargeable power source can be used. Because of the rapidity of the pulses required for modulating nerve tissue 54 (unlike cardiac pacing), there is a real need for power sources that will provide an acceptable service life under conditions of continuous delivery of high frequency pulses. FIG. 34A shows a graph of the energy density of several commonly used battery technologies. Lithium batteries have by far the highest energy density of commonly available batteries. Also, a lithium battery maintains a nearly constant voltage during discharge. This is shown in conjunction with FIG. 34B, which is normalized to the performance of the lithium battery. Lithium-ion batteries also have a long cycle life, and no memory effect. However, Lithium-ion batteries are not as tolerant to overcharging and overdischarging. One of the most recent development in rechargable battery technology is the Lithium-ion polymer battery. Recently the major battery manufacturers (Sony, Panasonic, Sanyo) have announced plans for Lithium-ion polymer battery production.

In another embodiment, existing nerve stimulators and cardiac pacemakers can be modified to incorporate rechargeable batteries. Among the nerve stimulators that can be adopted with rechargeable batteries can for, example, be the vagus nerve stimulator manufactured by Cyberonics Inc. (Houston, Tex.). U.S. Pat. No. 4,702,254 (Zabara), U.S. Pat. No. 5,023,807 (Zabara), and U.S. Pat. No. 4,867,164 (Zabara) on Neurocybernetic Prostheses, which can be practiced with rechargeable power source as disclosed in the next section. These patents are incorporated herein by reference.

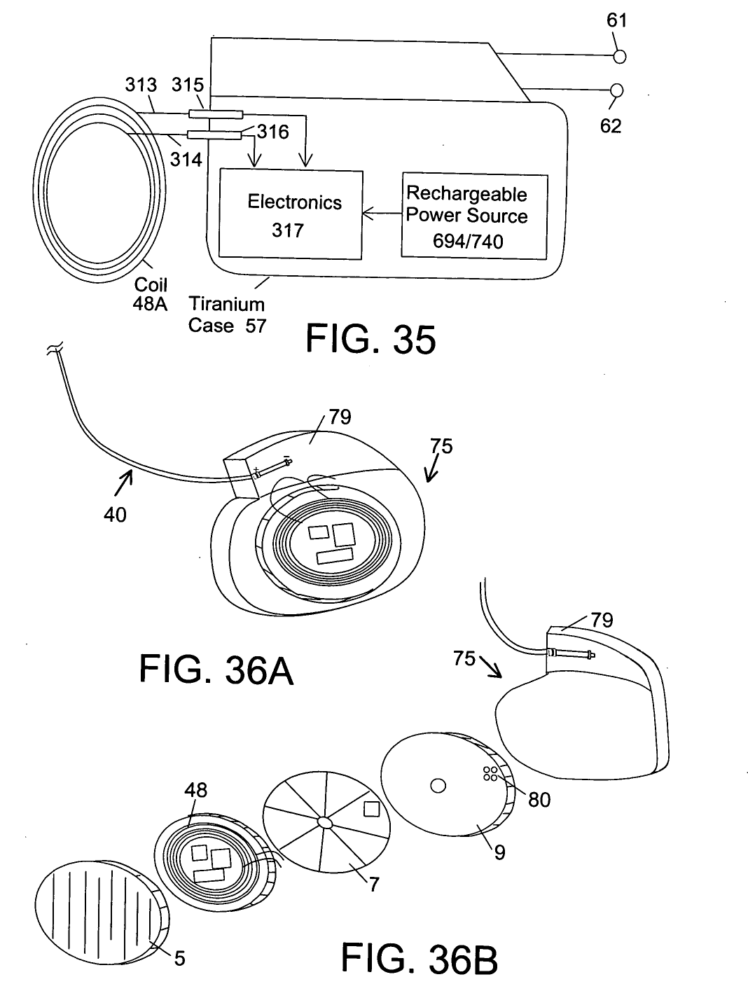

As shown in conjunction with FIG. 35, the coil is externalized from the titanium case 57. The RF pulses transmitted via coil 46 and received via subcutaneous coil 48A are rectified via a diode bridge. These DC pulses are processed and the resulting current applied to recharge the battery 694/740 in the implanted pulse generator. In one embodiment the coil 48C may be externalized at the header portion 79 of the implanted device, and may be wrapped around the titanium can, as was previously shown in FIGS. 28A-D.

In one embodiment, the coil may also be positioned on the titanium case as shown in conjunction with FIGS. 36A and 36B. FIG. 36A shows a diagram of the finished implantable stimulator 391R of one embodiment. FIG. 36B shows the pulse generator with some of the components used in assembly in an exploded view. These components include a coil cover 5, the secondary coil 48 and associated components, a magnetic shield 7, and a coil assembly carrier 9. The coil assembly carrier 9 has at least one positioning detail 80 located between the coil assembly and the feed through for positioning the electrical connection. The positioning detail 80 secures the electrical connection.

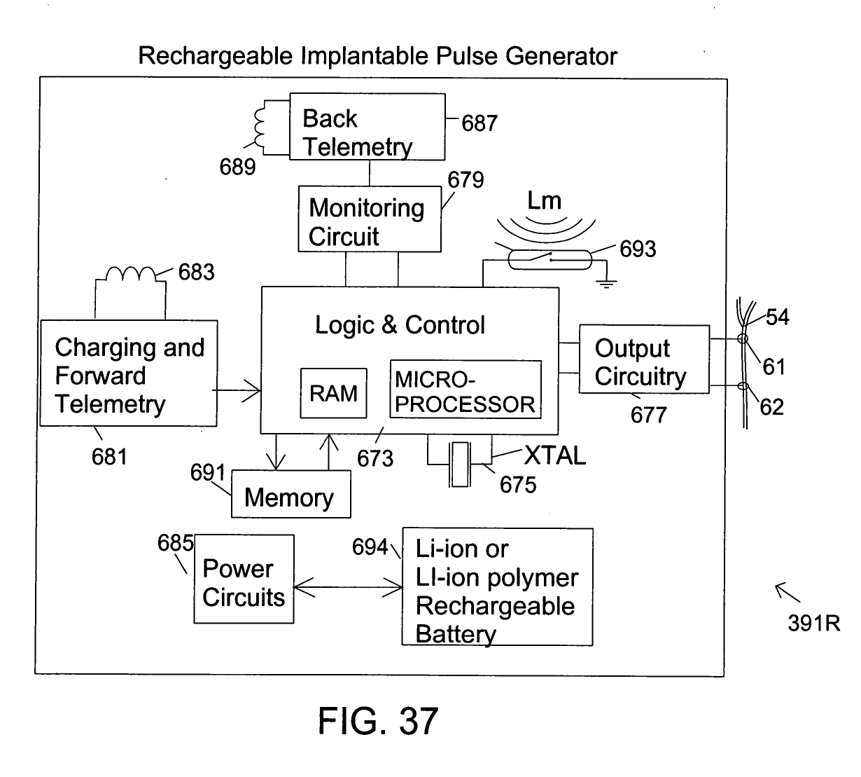

A schematic diagram of the implanted pulse generator (IPG 391R), with re-chargeable battery 694, is shown in conjunction with FIG. 37. The IPG 391R includes logic and control circuitry 673 connected to memory circuitry 691. The operating program and stimulation parameters are typically stored within the memory 691 via forward telemetry. Stimulation pulses are provided to the nerve tissue 54 via output circuitry 677 controlled by the microcontroller.

The operating power for the IPG 391R is derived from a rechargeable power source 694. The rechargeable power source 694 comprises a rechargeable lithium-ion or lithium-ion polymer battery. Recharging occurs inductively from an external charger to an implanted coil 48B underneath the skin 60. The rechargeable battery 694 may be recharged repeatedly as needed. Additionally, the IPG 391R is able to monitor and telemeter the status of its rechargable battery 691 each time a communication link is established with the external programmer 85.

Much of the circuitry included within the IPG 391R may be realized on a single application specific integrated circuit (ASIC). This allows the overall size of the IPG 391R to be quite small, and readily housed within a suitable hermetically-sealed case. The IPG case is preferably made from a titanium and is shaped in a rounded case.

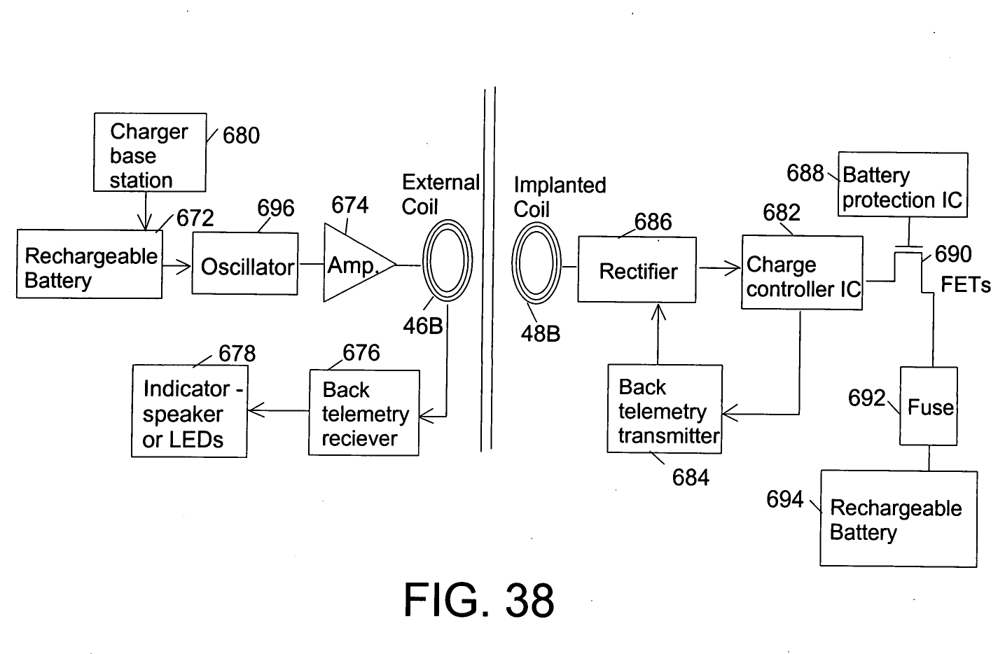

Shown in conjunction with FIG. 38 are the recharging elements of this embodiment. The re-charging system uses a portable external charger to couple energy into the power source of the IPG 391R. The DC-to-AC conversion circuitry 696 of the re-charger receives energy from a battery 672 in the re-charger. A charger base station 680 and conventional AC power line may also be used. The AC signals amplified via power amplifier 674 are inductively coupled between an external coil 46B and an implanted coil 48B located subcutaneously with the implanted pulse generator (IPG) 391R. The AC signal received via implanted coil 48B is rectified 686 to a DC signal which is used for recharging the rechargeable battery 694 of the IPG, through a charge controller IC 682. Additional circuitry within the IPG 391R includes, battery protection IC 688 which controls a FET switch 690 to make sure that the rechargeable battery 694 is charged at the proper rate, and is not overcharged. The battery protection IC 688 can be an off-the-shelf IC available from Motorola (part no. MC 33349N-3R1). This IC monitors the voltage and current of the implanted rechargeable battery 694 to ensure safe operation. If the battery voltage rises above a safe maximum voltage, the battery protection IC 688 opens charge enabling FET switches 690, and prevents further charging. A fuse 692 acts as an additional safeguard, and disconnects the battery 694 if the battery charging current exceeds a safe level. As also shown in FIG. 38, charge completion detection is achieved by a back-telemetry transmitter 684, which modulates the secondary load by changing the full-wave rectifier into a half-wave rectifier/voltage clamp. This modulation is in turn, sensed by the charger as a change in the coil voltage due to the change in the reflected impedance. When detected through a back telemetry receiver 676, either an audible alarm is generated or a LED is turned on.

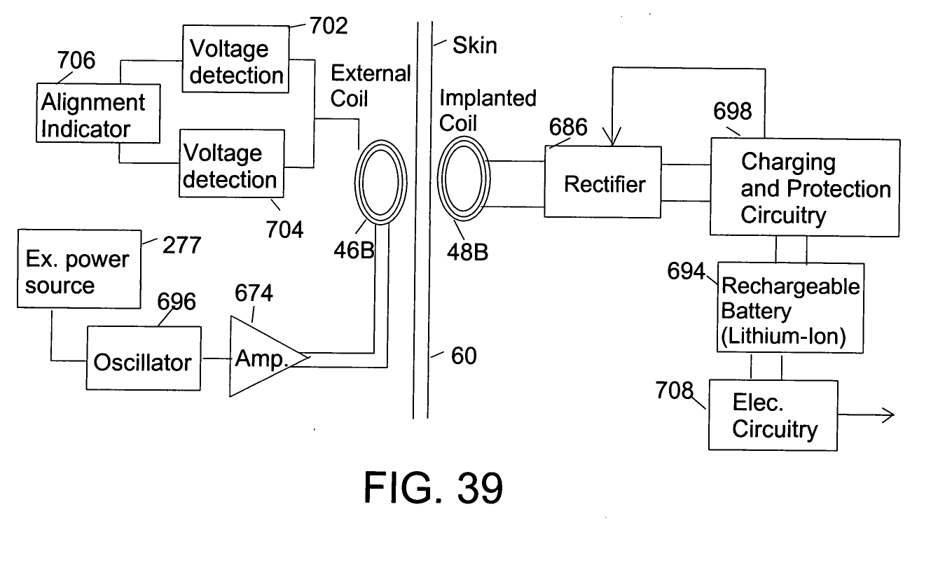

A simplified block diagram of charge completion and misalignment detection circuitry is shown in conjunction with FIG. 39. As shown, a switch regulator 686 operates as either a full-wave rectifier circuit or a half-wave rectifier circuit as controlled by a control signal (CS) generated by charging and protection circuitry 698. The energy induced in implanted coil 48B (from external coil 46B) passes through the switch rectifier 686 and charging and protection circuitry 698 to the implanted rechargeable battery 694. As the implanted battery 694 continues to be charged, the charging and protection circuitry 698 continuously monitors the charge current and battery voltage. When the charge current and battery voltage reach a predetermined level, the charging and protection circuitry 698 triggers a control signal. This control signal causes the switch rectifier 686 to switch to half-wave rectifier operation. When this change happens, the voltage sensed by voltage detector 702 causes the alignment indicator 706 to be activated. This indicator 706 may be an audible sound or a flashing LED type of indicator.

The indicator 706 may similarly be used as a misalignment indicator. In normal operation, when coils 46B (external) and 48B (implanted) are properly aligned, the voltage Vₛ sensed by voltage detector 704 is at a minimum level because maximum energy transfer is taking place. If and when the coils 46B and 48B become misaligned, then less than a maximum energy transfer occurs, and the voltage Vₛ sensed by detection circuit 704 increases significantly. If the voltage Vₛ reaches a predetermined level, alignment indicator 706 is activated via an audible speaker and/or LEDs for visual feedback. After adjustment, when an optimum energy transfer condition is established, causing Vₛ to decrease below the predetermined threshold level, the alignment indicator 706 is turned off.

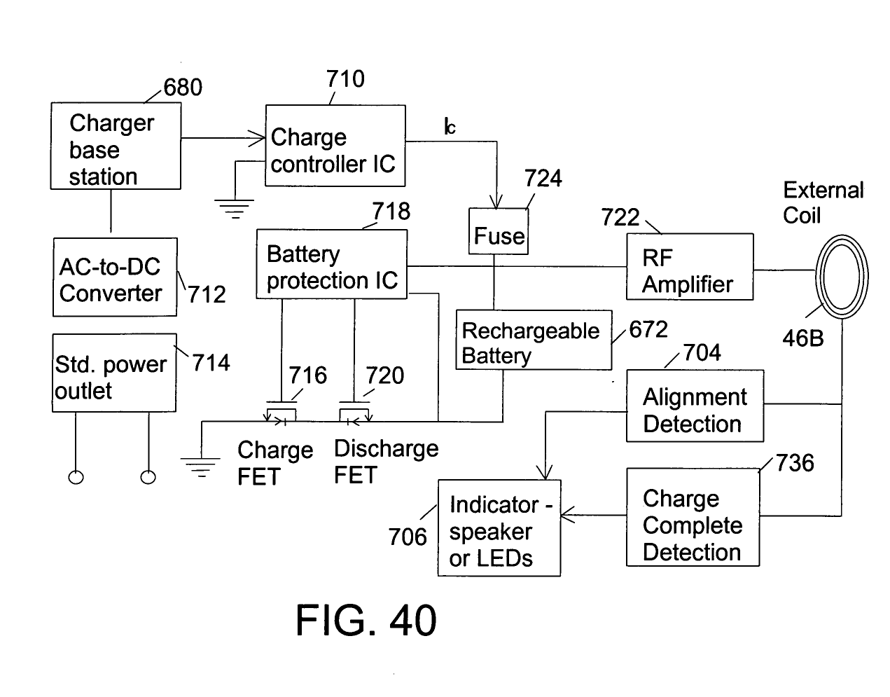

The elements of the external recharger are shown as a block diagram in conjunction with FIG. 40. In this disclosure, the words charger and recharger are used interchangeably. The charger base station 680 receives its energy from a standard power outlet 714, which is then converted to 5 volts DC by a AC-to-DC transformer 712. When the re-charger is placed in a charger base station 680, the re-chargeable battery 672 of the re-charger is fully recharged in a few hours and is able to recharge the battery 694 of the IPG 391R. If the battery 672 of the external re-charger falls below a prescribed limit of 2.5 volt DC, the battery 672 is trickle charged until the voltage is above the prescribed limit, and then at that point resumes a normal charging process.

As also shown in FIG. 40, a battery protection circuit 718 monitors the voltage condition, and disconnects the battery 672 through one of the FET switches 716, 720 if a fault occurs until a normal condition returns. A fuse 724 will disconnect the battery 672 should the charging or discharging current exceed a prescribed amount.

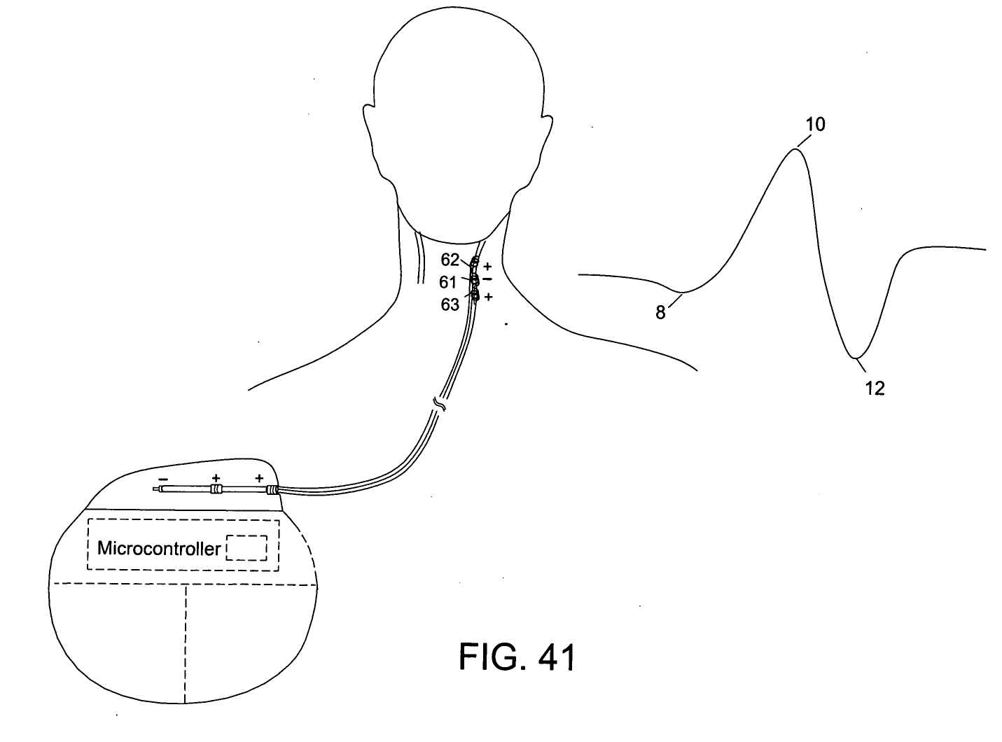

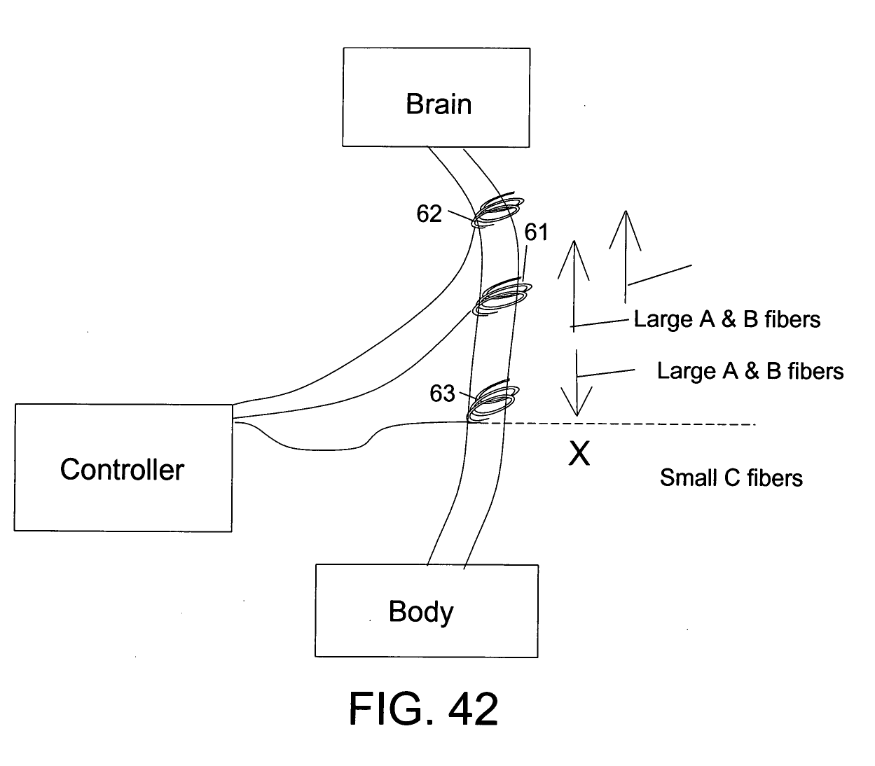

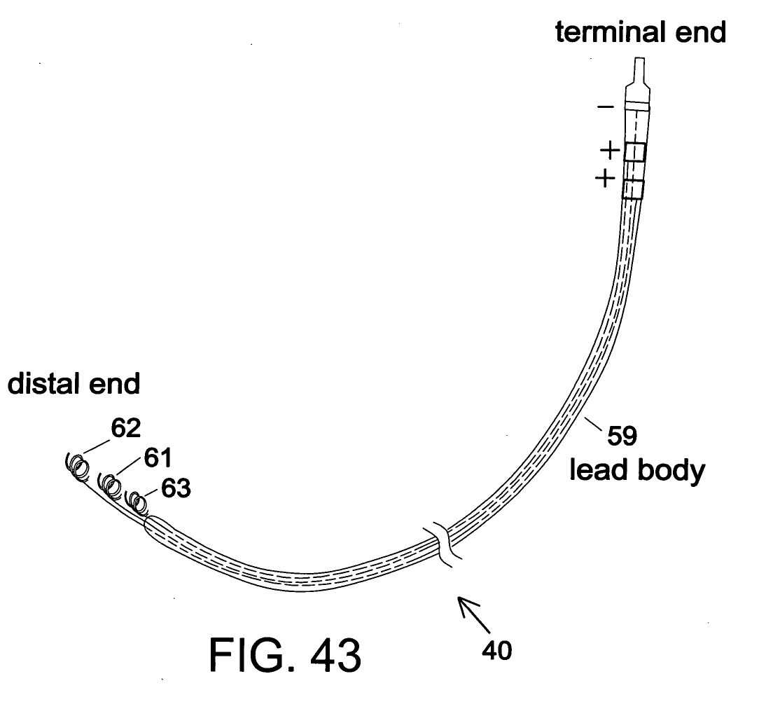

Since another key concept of this invention is to deliver afferent stimulation to vagus nerve(s), in one aspect efferent stimulation of selected types of fibers may be substantially blocked, utilizing the “greenwave” effect. In such a case, as shown in conjunction with FIGS. 41 and 42, a tripolar lead is utilized. As depicted on the top right portion of FIG. 41, there is a depolarization peak 10 on the vagus nerve bundle corresponding to electrode 61 (cathode) and the two hyper-polarization peaks 8, 12 corresponding to electrodes 62, 63 (anodes). With the microcontroller controlling the tripolar device, the size and timing of the hyper-polarizations 8, 12 can be controlled. Since the speed of conduction is different between the larger diameter A and B fibers and the smaller diameter c-fibers, by appropriately timing the pulses, collision blocks can be created for conduction via the large diameter A and B fibers in the efferent direction. This is depicted schematically in FIG. 42. A number of blocking techniques are known in the art, such as collision blocking, high frequency blocking, and anodal blocking. Any of these well known blocking techniques may be used with the practice of this invention, and are considered within the scope of this invention. A lead with tripolar electrodes for stimulation/blocking is shown in conjunction with FIG. 43.

In summary, in the method of the current invention for neuromodulation of cranial nerve such as the vagus nerve(s), to provide adjunct therapy along with ECT for severe depression can be practiced with any of the several pulse generator systems disclosed including,
a) an implanted stimulus-receiver with an external stimulator;
b) an implanted stimulus-receiver comprising a high value capacitor for storing charge, used in conjunction with an external stimulator;
c) a programmer-less implantable pulse generator (IPG) which is operable with a magnet;
d) a microstimulator;
e) a programmable implantable pulse generator;
f) a combination implantable device comprising both a stimulus-receiver and a programmable IPG; and
g) an IPG comprising a rechargeable battery.

Neuromodulation of vagus nerve(s) with any of these systems is considered within the scope of this invention.

In one embodiment, the external stimulator and/or the programmer has a telecommunications module, as described in a co-pending application, and summarized here for reader convenience. The telecommunications module has two-way communications capabilities.

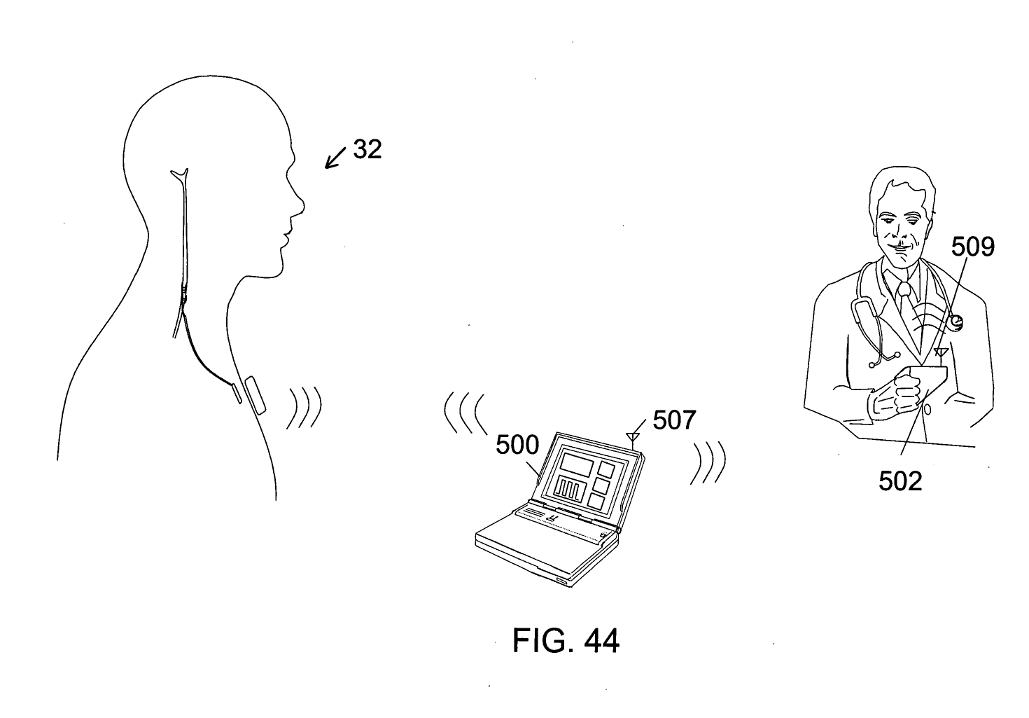

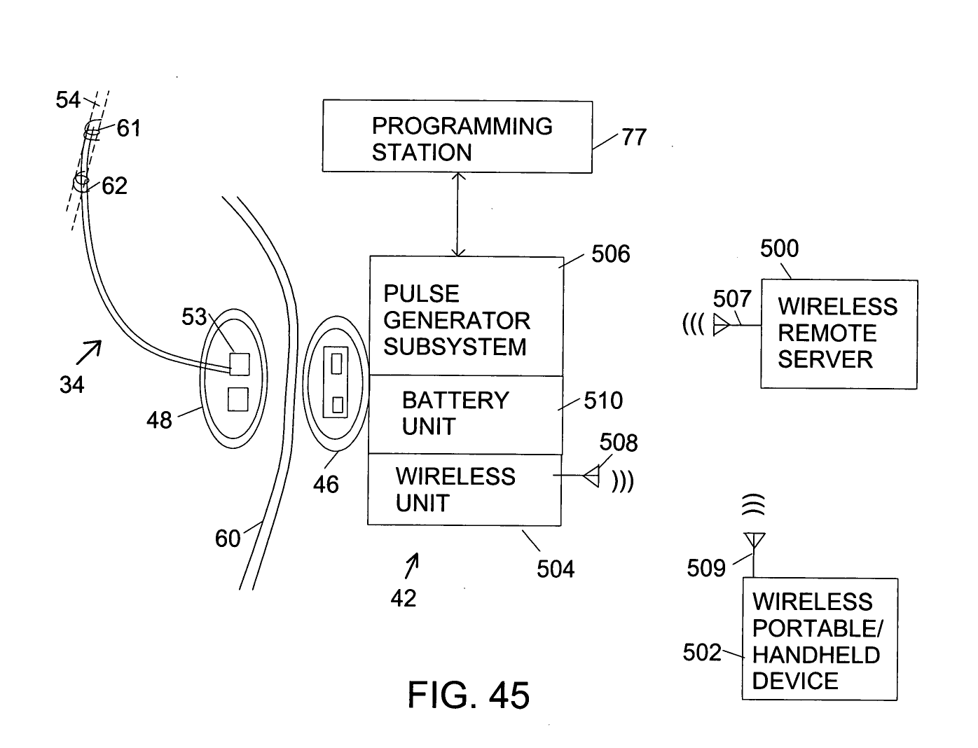

FIGS. 44 and 45 depict communication between an external stimulator 42 and a remote hand-held computer 502. A desktop or laptop computer can be a server 500 which is situated remotely, perhaps at a physician's office or a hospital. The stimulation parameter data can be viewed at this facility or reviewed remotely by medical personnel on a hand-held personal data assistant (PDA) 502, such as a “palm-pilot” from PALM corp. (Santa Clara, Calif.), a “Visor” from Handspring Corp. (Mountain view, Calif.) or on a personal computer (PC). The physician or appropriate medical personnel, is able to interrogate the external stimulator 42 device and know what the device is currently programmed to, as well as, get a graphical display of the pulse train. The wireless communication with the remote server 500 and hand-held PDA 502 would be supported in all geographical locations within and outside the United States (US) that provides cell phone voice and data communication service.

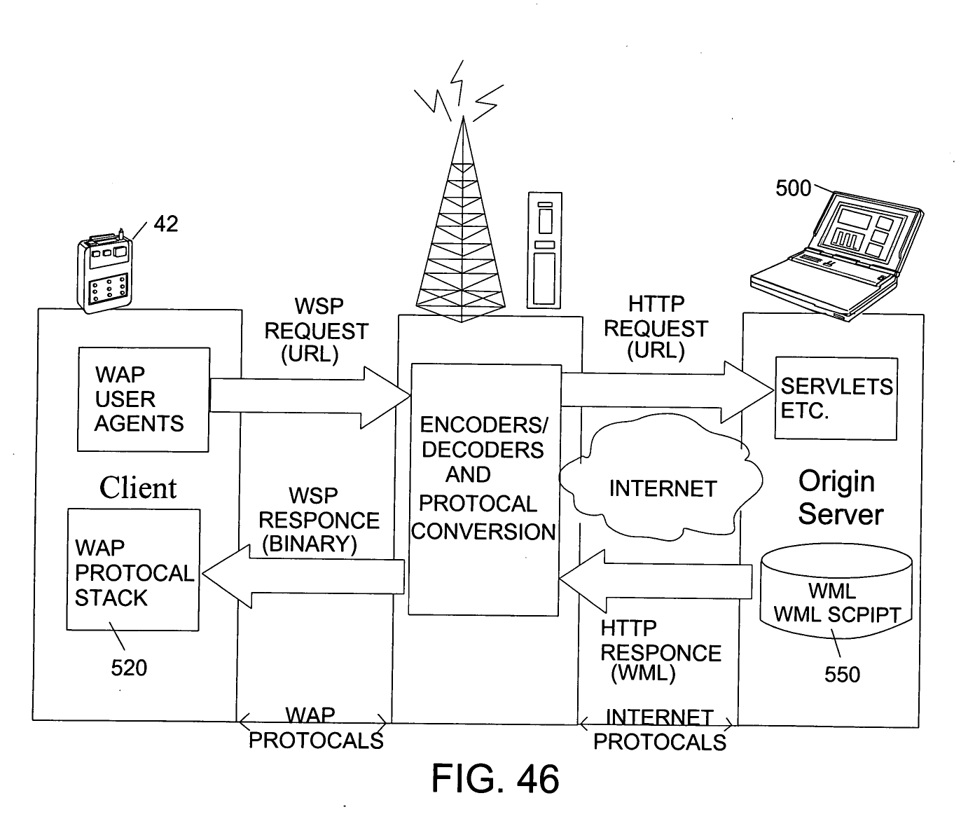

In one aspect of the invention, the telecommunications component can use Wireless Application Protocol (WAP). The Wireless Application Protocol (WAP), which is a set of communication protocols standardizing Internet access for wireless devices. While previously, manufacturers used different technologies to get Internet on hand-held devices, with WAP devices and services interoperate. WAP also promotes convergence of wireless data and the Internet. The WAP programming model is heavily based on the existing Internet programming model, and is shown schematically in FIG. 46. Introducing a gateway function provides a mechanism for optimizing and extending this model to match the characteristics of the wireless environment. Over-the-air traffic is minimized by binary encoding/decoding of Web pages and readapting the Internet Protocol stack to accommodate the unique characteristics of a wireless medium such as call drops.

The key components of the WAP technology, as shown in FIG. 46, includes 1) Wireless Mark-up Language (WML) 550 which incorporates the concept of cards and decks, where a card is a single unit of interaction with the user. A service constitutes a number of cards collected in a deck. A card can be displayed on a small screen. WML supported Web pages reside on traditional Web servers. 2) WML Script which is a scripting language, enables application modules or applets to be dynamically transmitted to the client device and allows the user interaction with these applets. 3) Microbrowser, which is a lightweight application resident on the wireless terminal that controls the user interface and interprets the WML/WMLScript content. 4) A lightweight protocol stack 520 which minimizes bandwidth requirements, guaranteeing that a broad range of wireless networks can run WAP applications. The protocol stack of WAP can comprise a set of protocols for the transport (WTP), session (WSP), and security (WTLS) layers. WSP is binary encoded and able to support header caching, thereby economizing on bandwidth requirements. WSP also compensates for high latency by allowing requests and responses to be handled asynchronously, sending before receiving the response to an earlier request. For lost data segments, perhaps due to fading or lack of coverage, WTP only retransmits lost segments using selective retransmission, thereby compensating for a less stable connection in wireless. The above mentioned features are industry standards adopted for wireless applications and greater details have been publicized, and well known to those skilled in the art.

In this embodiment, two modes of communication are possible. In the first, the server initiates an upload of the actual parameters being applied to the patient, receives these from the stimulator, and stores these in its memory, accessible to the authorized user as a dedicated content driven web page. The physician or authorized user can make alterations to the actual parameters, as available on the server, and then initiate a communication session with the stimulator device to download these parameters.

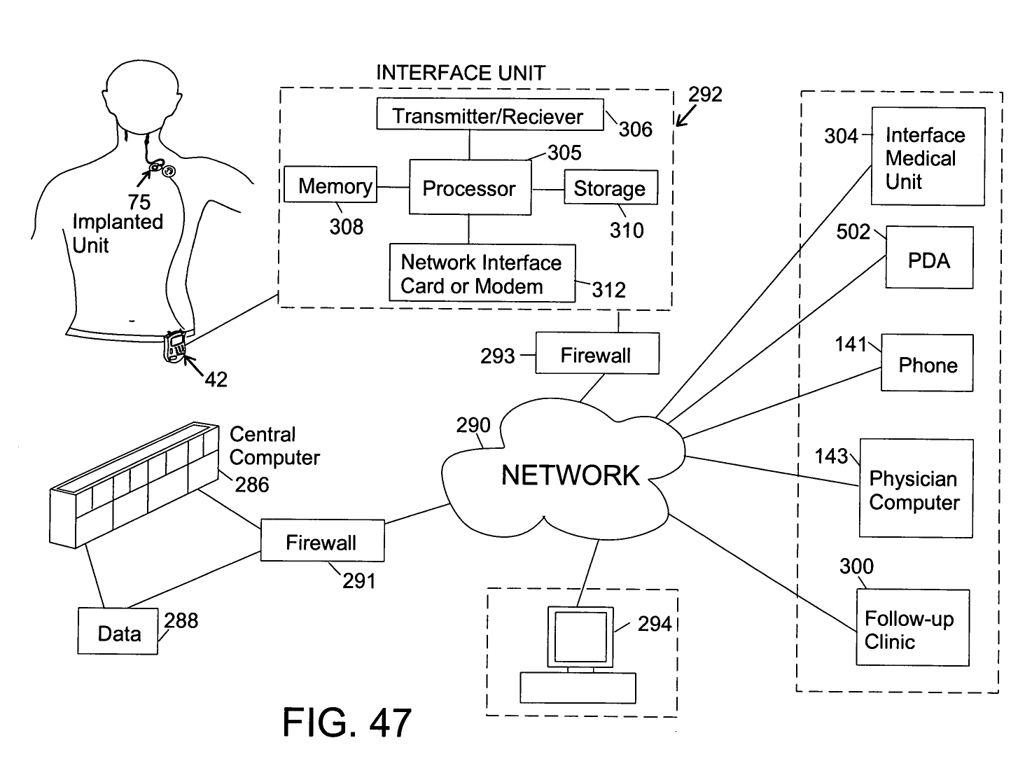

Shown in conjunction with FIG. 47, in one embodiment, the external stimulator 42 and/or the programmer 85 may also be networked to a central collaboration computer 286 as well as other devices such as a remote computer 294, PDA 502, phone 141, physician computer 143. The interface unit 292 in this embodiment communicates with the central collaborative network 290 via land-lines such as cable modem or wirelessly via the internet. A central computer 286 which has sufficient computing power and storage capability to collect and process large amounts of data, contains information regarding device history and serial number, and is in communication with the network 290. Communication over collaboration network 290 may be effected by way of a TCP/IP connection, particularly one using the internet, as well as a PSTN, DSL, cable modem, LAN, WAN or a direct dial-up connection.

The standard components of interface unit shown in block 292 are processor 305, storage 310, memory 308, transmitter/receiver 306, and a communication device such as network interface card or modem 312. In the preferred embodiment these components are embedded in the external stimulator 42 and can also be embedded in the programmer 85. These can be connected to the network 290 through appropriate security measures (Firewall) 293.

Another type of remote unit that may be accessed via central collaborative network 290 is remote computer 294. This remote computer 294 may be used by an appropriate attending physician to instruct or interact with interface unit 292, for example, instructing interface unit 292 to send instruction downloaded from central computer 286 to remote implanted unit.

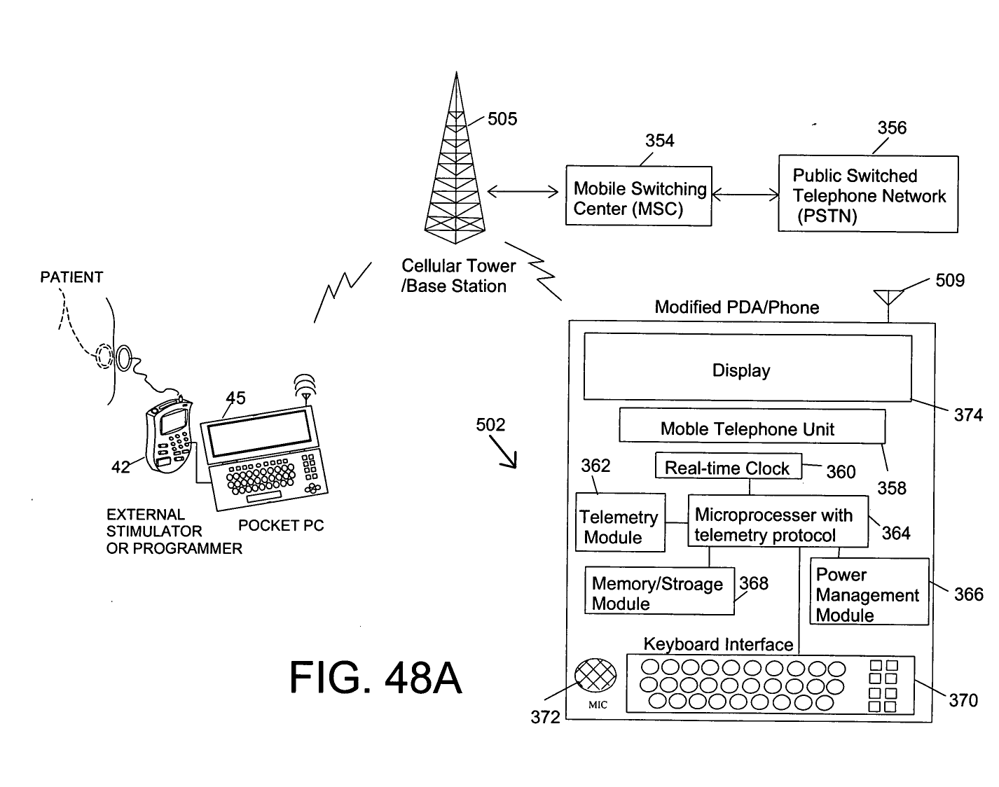

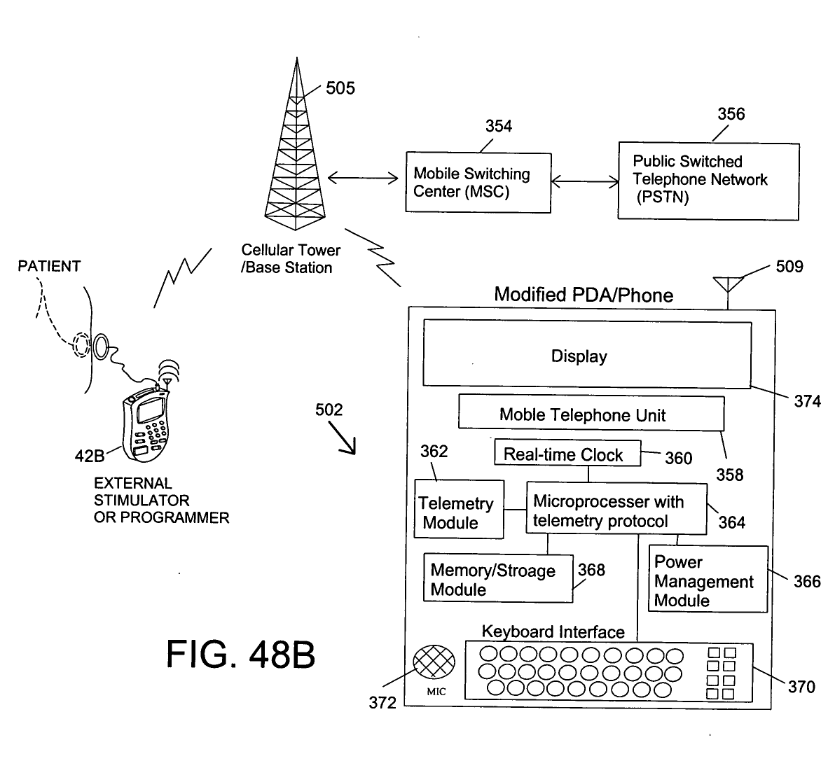

Shown in conjunction with FIGS. 48A and 48B the physician's remote communication's module is a Modified PDA/Phone 502 in this embodiment. The Modified PDA/Phone 502 is a microprocessor based device as shown in a simplified block diagram in FIGS. 65A and 65B. The PDA/Phone 502 is configured to accept PCM/CIA cards specially configured to fulfill the role of communication module 292 of the present invention. The Modified PDA/Phone 502 may operate under any of the useful software including Microsoft Window's based, Linux, Palm OS, Java OS, SYMBIAN, or the like.

The telemetry module 362 comprises an RF telemetry antenna 142 coupled to a telemetry transceiver and antenna driver circuit board which includes a telemetry transmitter and telemetry receiver. The telemetry transmitter and receiver are coupled to control circuitry and registers, operated under the control of microprocessor 364. Similarly, within stimulator a telemetry antenna 142 is coupled to a telemetry transceiver comprising RF telemetry transmitter and receiver circuit. This circuit is coupled to control circuitry and registers operated under the control of microcomputer circuit.

With reference to the telecommunications aspects of the invention, the communication and data exchange between Modified PDA/Phone 502 and external stimulator 42 operates on commercially available frequency bands. The 2.4-to-2.4853 GHz bands or 5.15 and 5.825 GHz, are the two unlicensed areas of the spectrum, and set aside for industrial, scientific, and medical (ISM) uses. Most of the technology today including this invention, use either the 2.4 or 5 GHz radio bands and spread-spectrum technology.

The telecommunications technology, especially the wireless internet technology, which this invention utilizes in one embodiment, is constantly improving and evolving at a rapid pace, due to advances in RF and chip technology as well as software development. Therefore, one of the intents of this invention is to utilize “state of the art” technology available for data communication between Modified PDA/Phone 502 and external stimulator 42. The intent of this invention is to use 3G technology for wireless communication and data exchange, even though in some cases 2.5G is being used currently.

For the system of the current invention, the use of any of the “3G” technologies for communication for the Modified PDA/Phone 502, is considered within the scope of the invention. Further, it will be evident to one of ordinary skill in the art that as future 4G systems, which will include new technologies such as improved modulation and smart antennas, can be easily incorporated into the system and method of current invention, and are also considered within the scope of the invention.

The present invention may be embodied in other specific forms without departing from the spirit or essential attributes thereof. It is therefore desired that the present embodiment be considered in all aspects as illustrative and not restrictive, reference being made to the appended claims rather than to the foregoing description to indicate the scope of the invention.

1. A method of providing electrical pulses to vagus nerve(s) and electroconvulsive therapy (ECT) to a patient to provide synergistic/addative benefits of said electrical pulses to vagus nerve(s) and electroconvulsive therapy (ECT) for treating or alleviating the symptoms of depression, comprising the steps of:
a) selecting a patient, wherein said patient is an electroconvulsive therapy patient, and
b) providing electrical pulses to vagus nerve(s), and/or its branches or part thereof,
whereby, said patient is provided said electroconvulsive therapy and vagus nerve(s) electrical stimulation.

2. Method of claim 1, wherein said electrical pulses are provided for stimulation and/or blocking of left or right vagus nerves or both, and/or its branches or part thereof in a patient.

3. Method of claim 1, wherein said depression comprises bipolar depression, unipolar depression, severe depression, treatment resistant depression, suicidal depression, psychotic depression, endogenous depression, and melancholia.

4. The method of claim 1, wherein said electroconvulsive therapy (ECT) provided to said patient and said electrical pulses provided to said vagus nerve(s), and/or its branches, or parts thereof are in any sequence, any combination, or any time intervals.

5. The method of claim 1, wherein said ECT comprises delivering electrical stimuli using brief-pulsed outputs at frequencies in the range of 30 Hz to 100 Hz.

6. The method of claim 1, wherein said said electric pulses to said vagus nerve(s) are provided by at least one pulse generator from a group consisting of: a) an implanted stimulus-receiver with an external stimulator; b) an implanted stimulus-receiver comprising a high value capacitor for storing charge, used in conjunction with an external stimulator; c) a programmer-less implantable pulse generator (IPG) which is operable with a magnet; d) a microstimulator; e) a programmable implantable pulse generator; f) a combination implantable device comprising both a stimulus-receiver and a programmable IPG; g) an IPG comprising a rechargeable battery.

7. The method of claim 1, wherein said electrical pulses provided to vagus nerve(s) have predetermined parameters, which can be programmed.

8. The method of claim 1, wherein said electrical pulses provided to said vagus nerve(s), and/or its branches, or parts thereof can be remotely controlled using a wide area network.

9. The method of claim 1, wherein said electroconvulsive therapy (ECT) and said electrical pulses to vagus nerve(s) are provided in addition to drug therapy.

10. A method of combining electroconvulsive therapy (ECT) and pulsed electrical stimulation to vagus nerve(s), and/or its branches or part thereof in a patient, for treating or alleviating the symptoms for at least one of depression, bipolar depression, unipolar depression, severe depression, treatment resistant depression, and melancholia, comprising the steps of:
a) selecting a depression patient;
b) providing electroconvulsive therapy to said patient; and
c) providing electrical pulses to said vagus nerve(s), and/or its branches or part thereof in said patient.

11. A method of claim 10, wherein said patient further comprises a patient who has received in past or is receiving or shall receive said electroconvulsive therapy (ECT).

12. The method of claim 10, wherein said electric stimulation to said vagus and/or its branches or part thereof nerve(s) is provided by at least one pulse generator from a group consisting of: a) an implanted stimulus-receiver with an external stimulator; b) an implanted stimulus-receiver comprising a high value capacitor for storing charge, used in conjunction with an external stimulator; c) a programmer-less implantable pulse generator (IPG) which is operable with a magnet; d) a microstimulator; e) a programmable implantable pulse generator; 0 a combination implantable device comprising both a stimulus-receiver and a programmable IPG; g) an IPG comprising a rechargeable battery.

13. The method of claim 10, wherein said electroconvulsive therapy (ECT) provided to said patient and said electrical pulses provided to said vagus nerve(s), and/or its branches, or parts thereof are in any sequence, any combination, or any time intervals.

14. A method of treating or alleviating the symptoms of depression by providing electrical pulses to vagus nerve(s), and/or its branches or part thereof and providing electrical pulses transcranially to the brain of a patient with electroconvulsive therapy (ECT), comprising the steps of:
a) selecting a patient;
b) providing electroconvulsive therapy to said patient; and
c) providing electrical pulses to vagus nerve(s), and/or its branches or part thereof in said patient.

15. The method of claim 14, wherein said electrical stimulation to said vagus nerve(s), and/or its branches or part thereof in a patient further comprises providing electric pulses to said vagus nerve for stimulation and/or blocking.

16. The method of claim 14, wherein said electroconvulsive therapy (ECT) provided to said patient and said electrical pulses provided to said vagus nerve(s), and/or its branches, or parts thereof are in any sequence, any combination, or any time intervals.

17. The method of claim 13, wherein said electric stimulation to said vagus and/or its branches or part thereof nerve(s) is provided by at least one pulse generator from a group consisting of: a) an implanted stimulus-receiver with an external stimulator; b) an implanted stimulus-receiver comprising a high value capacitor for storing charge, used in conjunction with an external stimulator; c) a magnet; d) a microstimulator; e) a programmable implantable pulse generator; f) a combination implantable device comprising both a programmer-less implantable pulse generator (IPG) which is operable with a stimulus-receiver and a programmable IPG; g) an IPG comprising a rechargeable battery.

18. A method of combining the therapeutic benefits of electroconvulsive therapy (ECT) and pulsed electrical stimulation to vagus nerve(s), and/or its branches or part thereof in a patient for treating or alleviating the symptoms for at least one of depression, bipolar depression, unipolar depression, severe depression, treatment resistant depression, and melancholia, comprising the steps of:
a) selecting a patient for providing said benefit;
b) providing electroconvulsive therapy (ECT), wherein said electroconvulsive therapy comprises placing at least one electrode means on patient's head and an external signal delivering means; and
c) providing electrical pulses to vagus nerve(s), comprising a lead with at least one electrode in contact with said vagus nerve and electrically connected to pulse generator means.

19. The method of claim 18, wherein said electroconvulsive therapy (ECT) and said electrical pulses to vagus nerve(s) are provided in addition to drug therapy.

20. The method of claim 18, wherein said electroconvulsive therapy (ECT) provided to said patient and said electrical pulses provided to said vagus nerve(s), and/or its branches, or parts thereof are in any sequence, any combination, or any time intervals.
